(19) United States
(12) Patent Application Publication
(10) Pub. No.: US 2026/0297201 A1
(43) Pub. Date: Oct. 1, 2026

(54) EGFR AND C-MET BINDING AGENT VARIANTS AND METHODS OF USE

(71) Applicant: Janssen Biotech, Inc, Horsham, PA (US)

(72) Inventors: Nuriye Berkay, Paoli, PA (US); Ping Hu, Fort Washington, PA (US); Qingrong Yan, Wayne, PA (US); Harsh Sharma, Malvern, PA (US)

(21) Appl. No.: 19/577,528

(22) Filed: Mar. 25, 2026

Related U.S. Application Data
(60) Provisional application No. 63/777,946, filed on Mar. 26, 2025.

(51) Int. Cl. C07K 16/28; C12N 9/26

(52) U.S. Cl. CPC C07K 16/2863; C12N 9/2474; C07K 2317/31; C07K 2317/732; C12Y 302/01035

(57) ABSTRACT

The present disclosure relates to EGFR/c-Met binding agent variants, compositions comprising EGFR/c-Met binding agent variants, and uses thereof.

### 1. CROSS-REFERENCE TO RELATED APPLICATIONS

This application claims the benefit of U.S. Provisional Application No. 63/777,946, filed on 26 Mar. 2025 which is incorporated herein by reference in its entirety.

### 2. SEQUENCE LISTING

The instant application contains a Sequence Listing, which has been submitted electronically in XML format and is hereby incorporated by reference in its entirety. Said XML copy, created on 23 Mar. 2026, is named JBI6987USNP1_SL.xml, and is 35,865 bytes in size.

### 3. FIELD

The present disclosure generally relates to bispecific agents that bind epidermal growth factor receptor (EGFR) and hepatocyte growth factor receptor (c-Met), compositions comprising EGFR/c-Met binding antibodies and variants thereof.

### 4. BACKGROUND

Amivantamab is a bispecific antibody that that binds epidermal growth factor receptor (EGFR) and hepatocyte growth factor receptor (c-Met). Amivantamab employs three distinct potential mechanisms of action (MOAs) including ligand blocking, receptor degradation, and immune cell-directing activity, such as antibody-dependent cellular cytotoxicity and trogocytosis. Through extensive characterization studies performed throughout the development of the product, it has been determined that biological function of the antibody may vary from batch to batch. Thus, a need exists for compositions suitable for maintaining the biological function of amivantamab and for treating EGFR and/or c-Met mediated diseases. Such compositions and related methods are provided by the present disclosure.

### 5. SUMMARY

In one aspect, provided herein is a composition comprising a variant EGFR/c-Met binding agent.

In some embodiments, the composition comprises a variant EGFR/c-Met binding agent comprising a first polypeptide comprising SEQ ID NO: 17, a second polypeptide comprising SEQ ID NO: 18, a third polypeptide comprising SEQ ID NO: 19, and a fourth polypeptide comprising SEQ ID NO: 20, wherein Asp99 of SEQ ID NO: 17 is isomerized, wherein the residue numbering starts from N-terminus of SEQ ID NO: 17, and wherein the composition further comprises a hyaluronidase.

In some embodiments, the composition further comprises a non-modified EGFR/c-Met binding agent comprising first polypeptide comprising SEQ ID NO: 17, a second polypeptide comprising SEQ ID NO: 18, a third polypeptide comprising SEQ ID NO: 19, and a fourth polypeptide comprising SEQ ID NO: 20.

In some embodiments, the composition facilitates binding to any one or more of EGFR and c-Met.

In some embodiments, the Asp99 isomerized variant of the EGFR/c-Met binding agent comprises from more than about 0% to about 10% of the EGFR/c-Met binding agents in the composition.

In some embodiments, the Asp99 isomerized variant EGFR/c-Met binding agent and the non-modified EGFR/c-Met binding agent have the fucose content between about 0% and about 20%.

In some embodiments, the hyaluronidase is a PH20.

In one aspect, provided herein is a pharmaceutical composition comprising a first polypeptide comprising SEQ ID NO: 17, a second polypeptide comprising SEQ ID NO: 18, a third polypeptide comprising SEQ ID NO: 19, and a fourth polypeptide comprising SEQ ID NO: 20, wherein Asp99 of SEQ ID NO: 17 is isomerized, wherein the residue numbering starts from N-terminus of SEQ ID NO: 17, and wherein the composition further comprises a hyaluronidase, and a pharmaceutically acceptable carrier.

In one aspect, provided herein is a method of killing a cancer or tumor cell, comprising contacting the cancer or tumor cell with an effective amount of the composition described herein.

In one aspect, provided herein is a method of killing a cancer or tumor cell in a subject in need thereof, comprising administering an effective amount of the composition described herein.

In one aspect, provided herein is a method of treating a cancer or tumor in a subject in need thereof, comprising administering an effective amount of the composition described herein.

In one aspect, provided herein is a method for treating a subject having a EGFR or c-Met-expressing cancer or tumor, comprising administering or providing for administration of an effective amount of the composition described herein to the subject.

In one aspect, the cancer or tumor is selected from lung cancer, colorectal cancer, and head and neck cancer.

In one aspect, the cancer or tumor is lung cancer.

In one aspect, the cancer or tumor is colorectal cancer.

In one aspect, the cancer or tumor is head and neck cancer.

### 6. DETAILED DESCRIPTION

The present disclosure provides compositions, comprising antibodies that bind epidermal growth factor receptor (EGFR) and hepatocyte growth factor receptor (c-Met), and variants thereof, and related subject matter.

### 6.1 General Techniques

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010).

### 6.2 Terminology

Unless described otherwise, all technical and scientific terms used herein have the same meaning as is commonly understood by one of ordinary skill in the art. For purposes of interpreting this specification, the following description of terms will apply and whenever appropriate, terms used in the singular will also include the plural and vice versa. All patents, applications, published applications, and other publications are incorporated by reference in their entirety. In the event that any description of terms set forth conflicts with any document incorporated herein by reference, the description of term set forth below shall control.

Unless otherwise defined herein, technical and scientific terms used in the present description have the meanings that are commonly understood by those of ordinary skill in the art. Whenever appropriate, terms used in the singular will also include the plural and vice versa. In the event that any description of a term set forth conflicts with any document incorporated herein by reference, the description of the term set forth below shall control.

As used herein, reference to “about” or “approximately” a value or parameter includes (and describes) embodiments that are directed to that value or parameter. As used herein, the term “about” used in connection with an amount indicates that the amount can vary by 10% of the stated amount. For example, “about 100” means an amount of from 90-110. Where “about” is used in the context of a range, “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

As used in the present disclosure and claims, the singular forms “a,” “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “binding agent” as used herein refers to a molecule that binds a specific antigen or target (e.g., EGFR and/or c-Met). A binding agent may comprise a protein, peptide, nucleic acid, carbohydrate, lipid, or small molecular weight compound. In some embodiments, a binding agent comprises a full-length antibody. In some embodiments, a binding agent is an antigen binding fragment of an antibody. In some embodiments, a binding agent comprises an alternative protein scaffold or artificial scaffold (e.g., a non-immunoglobulin backbone). In some embodiments, a binding agent is a fusion protein comprising an antigen-binding site. In some embodiments, a binding agent is a bispecific molecule comprising at least two antigen-binding sites. In some embodiments, a binding agent is a multispecific molecule comprising at least three antigen-binding sites.

The terms “Epidermal growth factor receptor” or “EGFR” as used herein refer to the human EGFR (also known as HER1 or ErbB1 (Ullrich et al., Nature 309:418-425, 1984) having the amino acid sequence shown in SEQ ID NO: 73 and in GenBank accession number NP_005219, as well as naturally-occurring variants thereof. Such variants include the well known EGFRvIII and other alternatively spliced variants (e.g., as identified by SwissProt Accession numbers P00533-1 (wild type; identical to SEQ ID NO: 73 and NP_005219), P00533-2 (F404L/L405S), P00533-3 (628-705:

CTGPGLEGCP . . . GEAPNQALLR→PGNESLKAML . . . SVIITASSCH and 706-1210 deleted), P00533-4 (C628S and 629-1210 deleted), variants GlnQ98, R266, K521, 1674, G962, and P988 (Livingston et al., NIEHS-SNPs, environmental genome project, NIEHS ES15478), T790M, L858R/T790M and del (E746, A750).

| PRT, Homo Sapiens, EGFR (includes signal sequence of 24 aa. Mature | |
|---|---|
| protein starts at residue 25), | |
| SEQ ID NO: 73 | |
| 1 | MRPSGTAGAA LLALLAALCP ASRALEEKKV CQGTSNKLTQ LGTFEDHFLS LQRMFNNCEV |
| 61 | VLGNLEITYV QRNYDLSFLK TIQEVAGYVL IALNTVERIP LENLQIIRGN MYYENSYALA |
| 121 | VLSNYDANKT GLKELPMRNL QEILHGAVRF SNNPALCNVE SIQWRDIVSS DFLSNMSMDF |
| 181 | QNHLGSCQKC DPSCPNGSCW GAGEENCQKL TKIICAQQCS GRCRGKSPSD CCHNQCAAGC |
| 241 | TGPRESDCLV CRKFRDEATC KDTCPPLMLY NPTTYQMDVN PEGKYSFGAT CVKKCPRNYV |
| 301 | VTDHGSCVRA CGADSYEMEE DGVRKCKKCE GPCRKVCNGI GIGEFKDSLS INATNIKHFK |
| 361 | NCTSISGDLH ILPVAFRGDS FTHTPPLDPQ ELDILKTVKE ITGFLLIQAW PENRTDLHAF |
| 421 | ENLEIIRGRT KQHGQFSLAV VSLNITSLGL RSLKEISDGD VIISGNKNLC YANTINWKKL |
| 481 | FGTSGQKTKI ISNRGENSCK ATGQVCHALC SPEGCWGPEP RDCVSCRNVS RGRECVDKCN |
| 541 | LLEGEPREFV ENSECIQCHP ECLPQAMNIT CTGRGPDNCI QCAHYIDGPH CVKTCPAGVM |
| 601 | GENNTLVWKY ADAGHVCHLC HPNCTYGCTG PGLEGCPTNG PKIPSIATGM VGALLLLLVV |
| 661 | ALGIGLFMRR RHIVRKRTLR RLLQERELVE PLTPSGEAPN QALLRILKET EFKKIKVLGS |
| 721 | GAFGTVYKGL WIPEGEKVKI PVAIKELREA TSPKANKEIL DEAYVMASVD NPHVCRLLGI |
| 781 | CLTSTVQLIT QLMPFGCLLD YVREHKDNIG SQYLLNWCVQ IAKGMNYLED RRLVHRDLAA |
| 841 | RNVLVKTPQH VKITDFGLAK LLGAEEKEYH AEGGKVPIKW MALESILHRI YTHQSDVWSY |
| 901 | GVTVWELMTF GSKPYDGIPA SEISSILEKG ERLPQPPICT IDVYMIMVKC WMIDADSRPK |
| 961 | FRELIIEFSK MARDPQRYLV IQGDERMHLP SPTDSNFYRA LMDEEDMDDV VDADEYLIPQ |
| 102 | QGFFSSPSTS RTPLLSSLSA TSNNSTVACI DRNGLQSCPI KEDSFLQRYS SDPTGALTED |
| 1081 | SIDDTFLPVP EYINQSVPKR PAGSVQNPVY HNQPLNPAPS RDPHYQDPHS TAVGNPEYLN |
| 1141 | TVQPTCVNST FDSPAHWAQK GSHQISLDNP DYQQDFFPKE AKPNGIFKGS TAENAEYLRV |
| 1201 | APQSSEFIGA |

The term “Hepatocyte growth factor receptor” or “c-Met” or “MET” as used herein refers to the human c-Met having the amino acid sequence shown in SEQ ID NO: 101 or in GenBank Accession No: NP_001120972 and natural variants thereof.

| SEQ ID NO: 101, PRT, Homo sapiens c-Met: | |
|---|---|
| 1 | mkapavlapg ilvllftlvq rsngeckeal aksemnvnmk yqlpnftaet piqnvilheh |
| 61 | hiflgatnyi yvlneedlqk vaeyktgpvl ehpdcfpcqd csskanlsgg vwkdninmal |
| 121 | vvdtyyddql iscgsvnrgt cqrhvfphnh tadiqsevhc ifspqieeps qcpdcvvsal |
| 181 | gakvlssvkd rfinffvgnt inssyfpdhp Ihsisvrrlk etkdgfmflt dqsyidvlpe |
| 241 | frdsypikyv hafesnnfiy fltvqretld aqtfhtriir fcsinsglhs ymemplecil |
| 301 | tekrkkrstk kevfnilqaa yvskpgaqla rqigaslndd ilfgvfaqsk pdsaepmdrs |
| 361 | amcafpikyv ndffnkivnk nnvrclqhfy gpnhehcfnr tllrnssgce arrdeyrtef |
| 421 | ttalqrvdlf mgqfsevllt sistfikgdl tianlgtseg rfmqvvvsrs gpstphvnfl |
| 481 | ldshpvspev ivehtlnqng ytlvitgkki tkiplnglgc rhfqscsqcl sappfvqcgw |
| 541 | chdkcvrsee clsgtwtqqi clpaiykvfp nsapleggtr Iticgwdfgf rrnnkfdlkk |
| 601 | trvllgnesc tltlsestmn tlkctvgpam nkhfnmsiii snghgttqys tfsyvdpvit |
| 661 | sispkygpma ggtlltltgn ylnsgnsrhi siggktctlk svsnsilecy tpaqtistef |
| 721 | avklkidlan retsifsyre dpivyeihpt ksfistwwke plnivsflfc fasggstitg |
| 781 | vgknlnsvsv prmvinvhea grnftvacqh rsnseiicct tpslqqlnlq lplktkaffm |
| 841 | ldgilskyfd liyvhnpvfk pfekpvmism gnenvleikg ndidpeavkg evlkvgnksc |
| 901 | enihlhseav lctvpndllk Inselniewk qaisstvlgk vivqpdqnft gliagvvsis |
| 961 | talllllgff lwlkkrkqik dlgselvryd arvhtphldr lvsarsvspt temvsnesvd |
| 1021 | yratfpedqf pnssqngscr qvqypltdms piltsgdsdi sspllqntvh idlsalnpel |
| 1081 | vqavqhvvig psslivhfne vigrghfgcv yhgtlldndg kkihcavksl nritdigevs |
| 1141 | qfltegiimk dfshpnvlsl lgiclrsegs plvvlpymkh gdlrnfirne thnptvkdli |
| 1201 | gfglqvakgm kylaskkfvh rdlaarncml dekftvkvad fglardmydk eyysvhnktg |
| 1261 | aklpvkwmal eslqtqkftt ksdvwsfgvl lwelmtrgap pypdvntfdi tvyllqgrrl |
| 1321 | lqpeycpdpl yevmlkcwhp kaemrpsfse lvsrisaifs tfigehyvhv natyvnvkcv |
| 1381 | apypsllsse dnaddevdtr pasfwets |

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the binding molecules described herein terms such as “bind to,” “that specifically bind to,” and analogous terms are also used interchangeably herein and refer to binding molecules of antigen binding domains that specifically bind to an antigen, such as a polypeptide. A binding molecule or antigen binding domain that binds to or specifically binds to an antigen can be identified, for example, by immunoassays, Octet®, Biacore®, or other techniques known to those of skill in the art. In some embodiments, a binding molecule or antigen binding domain binds to or specifically binds to an antigen when it binds to an antigen with higher affinity than to any cross-reactive antigen as determined using experimental techniques, such as enzyme linked immunosorbent assay (ELISA). Typically, a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of a binding molecule or antigen binding domain to a “non-target” protein is less than about 10% of the binding of the binding molecule or antigen binding domain to its particular target antigen, for example, as determined by fluorescence activated cell sorting (FACS) analysis. A binding molecule or antigen binding domain that binds to an antigen includes one that is capable of binding the antigen with sufficient affinity such that the binding molecule is useful, for example, as a therapeutic and/or diagnostic agent in targeting the antigen. In certain embodiments, a binding molecule or antigen binding domain that binds to an antigen has a dissociation constant (K_{D}) of less than or equal to 1 μM, 800 nM, 600 nM, 550 nM, 500 nM, 300 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, a binding molecule or antigen binding domain binds to an epitope of an antigen that is conserved among the antigen from different species.

The term “antibody,” “immunoglobulin,” or “Ig” is used interchangeably herein, and is used in the broadest sense and specifically covers, for example polyclonal antibodies, monoclonal antibodies (including agonist, antagonist, neutralizing antibodies, full-length monoclonal antibodies), antibody compositions with polyepitopic or monoepitopic specificity, recombinantly produced antibodies, single domain (e.g., VHH) antibodies, monospecific antibodies, multispecific antibodies (including bispecific antibodies), synthetic antibodies, chimeric antibodies, humanized antibodies, or human versions of antibodies having full-length heavy and/or light chains. Antibodies also include antibody fragments (and/or polypeptides that comprise antibody fragments) that retain binding characteristics of their parental antibodies. Non-limiting examples of antibody fragments include antigen-binding regions and/or effector regions of the antibody, e.g., Fab, Fab′, F(ab′)₂, Fv, scFv, (scFv)₂, single chain antibody molecule, dual variable domain antibody, single variable domain, linear antibody, V region, a multispecific antibody formed from antibody fragments, F(ab)₂, Fd, Fc, diabody, di-diabody, disulfide-linked Fvs (dsFv), single-domain antibody (e.g., nanobody) or other fragments (e.g., fragments consisting of the variable regions of the heavy and light chains that are non-covalently coupled). In general terms, a variable (V) region domain may be any suitable arrangement of immunoglobulin heavy (VH) and/or light (VL) variable domains. For example, antibodies also include tetrameric antibodies comprising two heavy chain and two light chain molecules, an antibody light chain monomer, and an antibody heavy chain monomer. Thus, for example, the V region domain may be dimeric and contain VHH-VHH, VH-VH, VH-VL, or VL-VL dimers that bind NKG2A. If desired, the VH and VL may be covalently coupled either directly or through a linker to form a single chain Fv (scFv). For ease of reference, scFv proteins are referred to herein as included in the category “antibody fragments.” Another form of an antibody fragment is a peptide comprising one or more complementarity determining regions (CDRs) of an antibody. CDRs (also termed “minimal recognition units” or “hypervariable regions”) can be obtained by constructing polynucleotides that encode one or more CDRs of interest. Such polynucleotides are prepared, for example, by using the polymerase chain reaction to synthesize the variable region using mRNA of antibody-producing cells as a template (see, for example, Larrick et al., Methods: A Companion to Methods in Enzymology, 2:106 (1991); Courtenay-Luck, “Genetic Manipulation of Monoclonal Antibodies,” in Monoclonal Antibodies Production, Engineering and Clinical Application, Ritter et al. (eds.), page 166, Cambridge University Press (1995); and Ward et al., “Genetic Manipulation and Expression of Antibodies,” in Monoclonal Antibodies: Principles and Applications, Birch et al., (eds.), page 137, Wiley-Liss, Inc. (1995)). Antibody fragments may be incorporated, for example, into single domain antibodies, maxibodies, minibodies, intrabodies, diabodies, triabodies, tetrabodies, variable domains of new antigen receptors (v-NAR), and bis-single chain Fv regions (see, e.g., Hollinger and Hudson, Nature Biotechnology, 23 (9): 1126-1136, 2005). In some embodiments, antibodies comprising a VH and/or VL contain a light chain and/or a heavy chain constant region, such as one or more constant regions, including one or more IgG1, IgG2, IgG3 and/or IgG4 constant regions. In some embodiments, antibodies can include epitope-binding fragments of any of the above. The antibodies described herein can be of any class (e.g., IgG, IgE, IgM, IgD, and IgA) or any subclass (e.g., IgG1, IgG2, IgG3, IgG4, IgA1, and IgA2) of immunoglobulin molecule.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions. A “functional fragment,” “binding fragment,” or “antigen binding fragment” of a therapeutic antibody will exhibit at least one if not some or all of the biological functions attributed to the intact antibody, the function comprising at least binding to the target antigen (e.g., a EGFR or c-Met binding fragment or fragment that binds to EGFR or c-Met).

A typical 4-chain antibody unit is a heterotetrametric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for u and & isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5th ed. 2001).

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat,” and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while μ and ε contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. CDR1, CDR2 and CDR3 in VH domain are also referred to as HCDR1, HCDR2 and HCDR3, respectively. CDR1, CDR2 and CDR3 in VL domain are also referred to as LCDR1, LCDR2 and LCDR3, respectively. Accordingly, CDRs are variable region sequences interspersed within the framework region sequences.

CDR regions are well known to those skilled in the art and have been defined by well-known numbering systems. For example, the Kabat Complementarity Determining Regions (CDRs) are based on sequence variability and are the most commonly used (see, e.g., Kabat et al., supra; Nick Deschacht et al., J Immunol 2010; 184:5696-5704). Chothia refers instead to the location of the structural loops (see, e.g., Chothia and Lesk, J. Mol. Biol. 196:901-17 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., Dev. Comp. Immunol. 27 (1): 55-77 (2003)). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309:657-70 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). The residues from each of these hypervariable regions or CDRs are exemplified in the table below.

Exemplary CDRs According to Various Numbering Systems

| Loop | Kabat | AbM | Chothia | Contact | IMGT |
|---|---|---|---|---|---|
| CDR L1 | L24--L34 | L24--L34 | L26--L32 or | L30--L36 | L27--L38 |
| | | | L24--L34 | | |
| CDR L2 | L50--L56 | L50--L56 | L50--L52 or | L46--L55 | L56--L65 |
| | | | L50--L56 | | |
| CDR L3 | L89--L97 | L89--L97 | L91--L96 or | L89--L96 | L105-L117 |
| | | | L89--L97 | | |
| CDR H1 | H31--H35B | H26--H35B | H26-- | H30--H35B | H27--H38 |
| | (Kabat | | H32 . . . 34 | | |
| | Numbering) | | | | |
| CDR H1 | H31--H35 | H26--H35 | H26--H32 | H30--H35 | |
| | (Chothia | | | | |
| | Numbering) | | | | |
| CDR H2 | H50--H65 | H50--H58 | H53--H55 or | H47--H58 | H56--H65 |
| | | | H52--H56 | | |
| CDR H3 | H95--H102 | H95--H102 | H96--H101 or | H93--H101 | H105-H117 |
| | | | H95--H102 | | |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the IMGT, Kabat, Chothia, or Contact method. In other cases, the particular amino acid sequence of a CDR is given. It should be noted CDR regions may also be defined by a combination of various numbering systems, e.g., a combination of Kabat and Chothia numbering systems, or a combination of Kabat and IMGT numbering systems. Therefore, the term such as “a CDR1 as set forth in a specific VH” includes any CDR1 as defined by the exemplary CDR numbering systems described above, but is not limited thereby. Once a variable region (e.g., a VH or VL) is given, those skilled in the art would understand that CDRs within the region can be defined by different numbering systems or combinations thereof.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

The term “antigen binding domain” or “antigen binding region” refers to a binding agent or a portion of a binding agent as described herein (such as a protein or an antibody or fragment thereof) that binds an antigen. In some embodiments, an antigen binding region can comprise one or more fragments or portions of an intact antibody as described herein. The term “antigen binding domain” or “antigen binding region” can be an antibody fragment as described above.

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail the sections below.

The term “single chain Fv” or “scFv” refers to a fusion protein comprising at least one antibody fragment comprising a light chain variable region (VL) and at least one antibody fragment comprising a heavy chain variable region (VH), wherein the VL and the VH are contiguously linked via a polypeptide linker, and capable of being expressed as a single chain polypeptide. Unless specified, as used herein, a scFv may have the VL and VH variable regions in either order, e.g., with respect to the N-terminal and C-terminal ends of the polypeptide, the scFv may comprise VL-linker-VH or may comprise VH-linker-VL.

The term “(scFv) 2” or “tandem scFv” or “bis-scFv” refers to a fusion protein comprising two light chain variable region (VL) and two heavy chain variable region (VH), wherein the two VL and the two VH are contiguously linked via polypeptide linkers, and capable of being expressed as a single chain polypeptide. The two VL and two VH are fused by peptide linkers to form a bivalent molecule VL_{A}-linker-VH_{A}-linker-VL_{B}-linker-VH_{B} to form two binding sites, capable of binding two different antigens or epitopes concurrently.

The term “multispecific” refers to a molecule, such as an antibody that specifically binds two or more distinct antigens or two or more distinct epitopes within the same antigen. Multispecific molecule may have cross-reactivity to other related antigens, for example to the same antigen from other species (homologs), such as human or monkey, for example Macaca fascicularis (cynomolgus, cyno) or Pan troglodytes, or may bind an epitope that is shared between two or more distinct antigens.

The term “bispecific” refers to a molecule (such as a protein or an antibody) that specifically binds two distinct antigens or two distinct epitopes within the same antigen. The bispecific molecule may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

The terms “EGFR/c-Met binding agent,” “bispecific anti-EGFR/anti-c-Met antibody,” “EGFR/c-Met antibody,” “EGFR×MET antibody,” “anti-EGFR/anti-c-Met protein,” and the like refer to an antibody that binds EGFR and/or c-Met, i.e., comprising at least one binding domain specifically binding EGFR and at least one binding domain specifically binding c-Met. The domains specifically binding EGFR and c-Met are typically V_{H}/V_{L} pairs. The bispecific anti-EGFR×c-Met antibody may be monovalent in terms of its binding to either EGFR or c-Met.

The term “monoclonal antibody” as used herein refers to a substantially homogenous antibody population involved in the highly specific recognition and binding of a single antigenic determinant or epitope. The term “monoclonal antibody” encompasses intact and full-length antibodies as well as antibody fragments (e.g., Fab, Fab′, F(ab′)₂, Fv), single chain antibodies, scFv, fusion proteins comprising an antigen-binding antibody fragment, and any other modified immunoglobulin molecule comprising at least one antigen-binding site. Furthermore, “monoclonal antibody” refers to such antibodies made by any number of techniques, including but not limited to, hybridoma production, phage library display, recombinant expression, and transgenic animals.

The term “Antibody-dependent cell-mediated cytotoxicity” or “ADCC” refers to a cell-mediated reaction in which non-specific cytotoxic cells that express Fc receptors (FcRs) (e.g. Natural Killer (NK) cells, neutrophils, and macrophages) recognize bound antibody on a target cell and subsequently cause lysis of the target cell.

The ability of monoclonal antibodies to induce ADCC can be enhanced by engineering their oligosaccharide component. Human IgG1 or IgG3 are N-glycosylated at Asn297 with the majority of the glycans in the well known biantennary G0, G0F, G1, G1F, G2 or G2F forms. Antibodies produced by non-engineered CHO cells typically have a glycan fucose content of about at least 85%. The removal of the core fucose from the biantennary complex-type oligosaccharides attached to the Fc regions enhances the ADCC of antibodies via improved FcγRIIIa binding without altering antigen binding or CDC activity. Such mAbs can be achieved using different methods reported to lead to the successful expression of relatively high defucosylated antibodies bearing the biantennary complex-type of Fc oligosaccharides such as control of culture osmolality (Konno et al., Cytotechnology 64(:249-65, 2012), application of a variant CHO line Lec13 as the host cell line (Shields et al., J Biol Chem 277:26733-26740, 2002), application of a variant CHO line EB66 as the host cell line (Olivier et al., MAbs; 2 (4), 2010; Epub ahead of print; PMID: 20562582), application of a rat hybridoma cell line YB2/0 as the host cell line (Shinkawa et al., J Biol Chem 278:3466-3473, 2003), introduction of small interfering RNA specifically against the alpha-1,6-fucosyltrasferase (FUT8) gene (Mori et al., Biotechnol Bioeng88:901-908, 2004), or coexpression of beta-1,4-N-acetylglucosaminyltransferase III and Golgi α-mannosidase II or a potent alpha-mannosidase I inhibitor, kifunensine (Ferrara et al., J Biol Chem 281:5032-5036, 2006, Ferrara et al., Biotechnol Bioeng 93:851-861, 2006; Xhou et al., Biotechnol Bioeng 99:652-65, 2008).

In some embodiments described herein, ADCC elicited by the bispecific EGFR/c-Met antibodies may also be enhanced by certain substitutions in the antibody Fc. Exemplary substitutions are for example substitutions at amino acid positions 256, 290, 298, 312, 356, 330, 333, 334, 360, 378 or 430 (residue numbering according to the EU index) as described in U.S. Pat. No. 6,737,056.

“Fucose content” means the amount of the fucose monosaccharide within the sugar chain at Asn297. The relative amount of fucose is the percentage of fucose-containing structures related to all glycostructures. These may be characterized and quantified by multiple methods, for example: 1) using MALDI-TOF of N-glycosidase F treated sample (e.g. complex, hybrid and oligo- and high-mannose structures) as described in Int Pat. Publ. No. WO2008/077546 2); 2) by enzymatic release of the Asn297 glycans with subsequent derivatization and detection/quantitation by HPLC (UPLC) with fluorescence detection and/or HPLC-MS (UPLC-MS); 3) intact protein analysis of the native or reduced mAb, with or without treatment of with Endo S or other enzymes to remove N-linked glycosylation; 4) digestion of the mAb to constituent peptides by enzymatic digestion (e.g., trypsin or endopeptidase Lys-C), and subsequent separation, detection and quantitation by HPLC-MS (UPLC-MS); 5) Separation of the mAb oligosaccharides from the mAb protein by specific enzymatic deglycosylation with PNGase F at Asn 297. The oligosaccharides thus released can be labeled with a fluorophore, separated and identified by various complementary techniques which allow: fine characterization of the glycan structures by matrix-assisted laser desorption ionization (MALDI) mass spectrometry by comparison of the experimental masses with the theoretical masses, determination of the degree of sialylation by ion exchange HPLC (GlycoSep C), separation and quantification of the oligosacharride forms according to hydrophilicity criteria by normal-phase HPLC (GlycoSep N), and separation and quantification of the oligosaccharides by high performance capillary electrophoresis-laser induced fluorescence (HPCE-LIF).

“Normal fucose” or ‘normal fucose content” as used herein refers to antibodies with fucose content of about over 50%, typically about over 80% or over 85%.

The terms “epitope” and “antigenic determinant” are used interchangeably herein and refer to that portion of an antigen or target capable of being recognized and bound by a particular antibody. When the antigen or target is a polypeptide, epitopes can be formed both from contiguous amino acids and noncontiguous amino acids juxtaposed by tertiary folding of the protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation. Epitopes can be predicted using any one of a large number of publicly available bioinformatic software tools. X-ray crystallography may be used to characterize an epitope on a target protein by analyzing the amino acid residue interactions of an antigen/antibody complex.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies, the term “polypeptide” encompasses polypeptides as a single chain and polypeptides of two or more associated chains.

The terms “polynucleotide” and “nucleic acid” and “nucleic acid molecule” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the disclosure are substantially identical, meaning they have at least 70%, at least 75%, at least 80%, at least 85%, at least 90%, and in some embodiments at least 95%, 96%, 97%, 98%, 99% nucleotide or amino acid identity, when compared and aligned for maximum correspondence, as measured using a sequence comparison algorithm or by visual inspection. In some embodiments, identity exists over a region of the sequences that is at least about 10, at least about 20, at least about 20-40, at least about 40-60, at least about 60-80 nucleotides or amino acids in length, or any integral value there between. In some embodiments, identity exists over a longer region than 60-80 nucleotides or amino acids, such as at least about 80-100 nucleotides or amino acids, and in some embodiments the sequences are substantially identical over the full length of the sequences being compared, for example, (i) the coding region of a nucleotide sequence or (ii) an amino acid sequence.

The term “vector” as used herein means a construct that is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

The term “isolated” as used herein refers to a polypeptide, soluble protein, antibody, polynucleotide, vector, cell, or composition that is in a form not found in nature. An “isolated” antibody is substantially free of material from the cellular source from which it is derived. In some embodiments, isolated polypeptides, soluble proteins, antibodies, polynucleotides, vectors, cells, or compositions are those that have been purified to a degree that they are no longer in a form in which they are found in nature. In some embodiments, a polypeptide, soluble protein, antibody, polynucleotide, vector, cell, or composition that is isolated is substantially pure. A polypeptide, soluble protein, antibody, polynucleotide, vector, cell, or composition can be isolated from a natural source (e.g., tissue) or from a source such as an engineered cell line.

The term “substantially pure” as used herein refers to material that is at least 50% pure (i.e., free from contaminants), at least 90% pure, at least 95% pure, at least 98% pure, or at least 99% pure.

The term “subject” refers to any animal (e.g., a mammal), including, but not limited to, humans, non-human primates, canines, felines, rabbits, rodents, and the like.

The term “excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle. In some embodiments, excipients are pharmaceutically acceptable excipients. Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™ polyethylene glycol (PEG), and PLURONICS™. Other examples of pharmaceutically acceptable excipients are described in Remington and Gennaro, Remington's Pharmaceutical Sciences (18th ed. 1990). In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution. In some embodiments, excipients are sterile liquids, such as water and oils, including those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. An excipient can also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard excipients such as pharmaceutical grades of mannitol, lactose, starch, magnesium stearate, sodium saccharine, cellulose, magnesium carbonate, etc. Compositions, including pharmaceutical compounds, may contain a prophylactically or therapeutically effective amount of a NKG2A binding agent (e.g., an antibody), for example, in isolated or purified form, together with a suitable amount of excipient so as to provide the form for proper administration to the subject (e.g., patient). The formulation should suit the mode of administration.

The term “pharmaceutical composition” or “pharmaceutical formulation” as used herein refers to a preparation that is in such form as to permit the biological activity of the binding agent to be effective. A pharmaceutical formulation or composition generally comprises additional components, such as a pharmaceutically acceptable excipient, carrier, adjuvant, buffers, etc.

The term “effective amount” or “therapeutically effective amount” as used herein refers to the amount of an agent that is sufficient to reduce and/or ameliorate the severity and/or duration of (i) a disease, disorder or condition in a subject, and/or (ii) a symptom in a subject. The term also encompasses an amount of an agent necessary for the (i) reduction or amelioration of the advancement or progression of a given disease, disorder, or condition, (ii) reduction or amelioration of the recurrence, development, or onset of a given disease, disorder, or condition, and/or (iii) the improvement or enhancement of the prophylactic or therapeutic effect(s) of another agent or therapy (e.g., an agent other than the binding agents provided herein).

The term “treat” or “treatment” or “treating” or “to treat” or “alleviate” or alleviation” or “alleviating” or “to alleviate” as used herein refers to therapeutic measures that aim to cure, slow down, lessen symptoms of, and/or halt progression of a pathologic condition or disorder. Thus, those in need of treatment include those already with the disorder.

The term “immune response” as used herein includes responses from both the innate immune system and the adaptive immune system. It includes both cell-mediated and/or humoral immune responses. It includes both T-cell and B-cell responses, as well as responses from other cells of the immune system such as natural killer (NK) cells, monocytes, macrophages, dendritic cells, etc.

“Hyaluronidase” refers to a class of enzymes that degrade hyaluronan. Hyaluronidases are endoglycosidases used to increase the dispersion and absorption of other co-administered drugs when administered subcutaneously (e.g., subcutaneous injections, subcutaneous infusion such as hypodermoclysis). Hyaluronidases include, but are not limited to, bacterial hyaluronidases (EC 4.2.2.1 or EC 4.2.99.1), hyaluronidases from leeches, other parasites and crustaceans (EC 3.2.1.36), and mammalian-type hyaluronidases (EC 3.2.1.35). Hyaluronidase (recombinant human) has a molecular weight of approximately 61 kDa. Hyaluronidases include those of non-human origin including, but not limited to, murine, canine, feline, leporine, avian, bovine, ovine, porcine, equine, piscine, ranine, bacterial, and any from leeches, other parasites, and crustaceans. Exemplary human hyaluronidases include HYAL1, HYAL2, HYAL3, HYAL4, and PH20. Also included amongst hyaluronidases are soluble hyaluronidases, including, ovine and bovine PH20, and soluble forms of PH20. Exemplary hyaluronidases include those set forth in U.S. Pub. No. 2013/0302275, which is incorporated by reference herein, including, for example, hyaluronidases set forth as SEQ ID NOs: SEQ ID NOS: 6, 7-31, 69, 70, 71, 72, 856-861, 869-921 from U.S. Pub. No. 2013/0302275 (which are incorporated herein by reference), mature forms thereof (lacking the signal sequence), allelic variants thereof, and truncated forms thereof that exhibit hyaluronidase activity, including C-terminal truncated variants that are soluble.

As used herein, PH20 refers to a type of hyaluronidase that occurs in sperm and is neutral-active.

Soluble PH20 refers to a polypeptide characterized by its solubility under physiological conditions. Generally, a soluble PH20 lacks all or a portion of a glycophosphatidyl anchor (GPI) attachment sequence, or does not otherwise sufficiently anchor to the cell membrane. For example, a soluble PH20 can be a C-terminally truncated variant of a PH20 lacking a contiguous sequence of amino acids that corresponds to all or a portion of a GPI anchor attachment sequence. Hence, upon expression from a cell, a soluble PH20 is secreted into the medium.

Soluble human PH20 (sHuPH20) includes human PH20 polypeptides that lack a contiguous sequence of amino acids from the C-terminus of human PH20 that includes all or a portion of the GPI anchor sequence (C-terminally truncated PH20 polypeptides) such that upon expression, the polypeptides are soluble under physiological conditions. For example, soluble human PH20 polypeptides are C-terminally truncated polypeptides of human PH20 in its precursor form or in its mature form lacking the signal sequence, or allelic variants thereof as set forth in SEQ ID NOs: 26-32 herein and as disclosed in U.S. Pub. No. 20130302275 as SEQ ID NOs: 6, 7, and 68-72 of that reference which are incorporated herein by reference.

U.S. Pub. No. 2004/0268425, which is incorporated by reference herein, describes members of the soluble, neutral active Hyaluronidase Glycoprotein family, particularly the human soluble PH-20 Hyaluronidase Glycoproteins (also referred to as sHASEGPs). U.S. Pub. No. 20100143457, which is incorporated herein by reference, describes shorter active soluble PH20 (i.e., 36-469, -470, 471, and longer forms i.e., 36-495 to 36-500) the sequences of which are also incorporated herein by reference.

Hyaluronidase activity refers to the ability to enzymatically catalyze the cleavage of hyaluronic acid. The United States Pharmacopeia (USP) XXII assay for hyaluronidase determines hyaluronidase activity indirectly by measuring the amount of higher molecular weight hyaluronic acid, or hyaluronan, (HA) substrate remaining after the enzyme is allowed to react with the HA for 30 min at 37° C. (USP XXII-NF XVII (1990) 644-645 United States Pharmacopeia Convention, Inc, Rockville, MD). A Reference Standard solution can be used in an assay to ascertain the relative activity, in units, of any hyaluronidase. In vitro assays to determine the hyaluronidase activity of hyaluronidases, such as PH20, including modified PH20 polypeptides, are known in the art and described herein. Exemplary assays include the microturbidity assay that measures cleavage of hyaluronic acid by hyaluronidase indirectly by detecting the insoluble precipitate formed when the uncleaved hyaluronic acid binds with serum albumin. Reference Standards can be used, for example, to generate a standard curve to determine the activity in Units of the hyaluronidase being tested.

Specific activity refers to Units of activity per mg protein. The milligrams of hyaluronidase is defined by the absorption of a solution of at 280 nm assuming a molar extinction coefficient of approximately 1.7, in units of M−1 cm−1.

Neutral active refers to the ability of a PH20 polypeptide to enzymatically catalyze the cleavage of hyaluronic acid at neutral pH, such as at a pH between or about between pH 6.0 to pH 7.8.

Human recombinant DNA-derived hyaluronidase enzyme PH20 (rHuPH20) is a glycosylated single-chain protein produced by CHO cells containing a DNA plasmid encoding residues 36-482 of SEQ ID NO: 26, resulting in a heterogeneous mixture of soluble forms of human hyaluronidase (PH20) that begin at residue 36 and terminate at residues 478, 479, 480, 481, and 482 of SEQ ID NO: 26, including any one of SEQ ID NOs: 21, 25, 24, 23 and 22.

### 6.3 EGFR/c-Met Binding Agents

Amivantamab is a bispecific antibody that that binds epidermal growth factor receptor (EGFR) and hepatocyte growth factor receptor (c-Met). Amivantamab employs three distinct potential mechanisms of action (MOAs) including ligand blocking, receptor degradation, and immune cell-directing activity, such as antibody-dependent cellular cytotoxicity and trogocytosis. Through extensive characterization studies performed throughout the development of the product, it has been determined that some post-translational modifications (PTMs), for example isomerization and fucosylation levels are critical quality attributes of amivantamab. Importantly, it has been found that specific levels of these variants must be maintained to ensure appropriate biological function.

In some aspects, the exemplary bispecific EGFR/c-Met antibody comprises a first heavy chain (HC1) comprising a HC1 variable region 1 (VH1); a first light chain (LC1) comprising a light chain variable region 1 (VL1); a second heavy chain (HC2) comprising a HC2 variable region 2 (VH2); and a second light chain (LC2) comprising a light chain variable region 2 (VL2), wherein the VH1 comprises a heavy chain complementarity determining region 1 (HCDR1), a HCDR2 and a HCDR3 amino acid sequences of SEQ ID NOs: 1, 2, and 3, respectively; the VL1 comprises a light chain complementarity determining region 1 (LCDR1), a LCDR2 and a LCDR3 amino acid sequences of SEQ ID NOs: 4, 5 and 6, respectively; the VH2 comprises the HCDR1, the HCDR2 and the HCDR3 amino acid sequences of SEQ ID NOs: 7, 8 and 9, respectively; and the VL2 comprises the LCDR1, the LCDR2 and the LCDR3 amino acid sequences of SEQ ID NOs: 10, 11 and 12, respectively.

In some embodiments, the first heavy chain (HC1) of the bispecific EGFR-c-Met antibody comprises a HC1 constant domain 3 (HC1 CH3) and a HC1 variable region 1 (VH1). In some embodiments, the second heavy chain (HC2) of the bispecific EGFR-c-Met antibody comprises a HC2 constant domain 3 (HC2 CH₃) and a HC2 variable region 2 (VH2). In some embodiments, the first heavy chain (HC1) of the bispecific EGFR-c-Met antibody comprises a HC1 constant domain 3 (HC1 CH3) and a HC1 variable region 1 (VH1 and the second heavy chain (HC2) of the bispecific EGFR-c-Met antibody comprises a HC2 constant domain 3 (HC2 CH3) and a HC2 variable region 2 (VH2). In some embodiments, the first heavy chain (HC1) of the bispecific EGFR-c-Met antibody comprises a HC1 constant domain 2 and constant domain 3 (HC1 CH2-CH3) and a HC1 variable region 1 (VH1). In some embodiments, the second heavy chain (HC2) of the bispecific EGFR-c-Met antibody comprises a HC2 constant domain 2 and constant domain 3 (HC2 CH2-CH3) and a HC2 variable region 2 (VH2). In some embodiments, the first heavy chain (HC1) of the bispecific EGFR-c-Met antibody comprises a HC1 constant domain 2 and constant domain 3 (HC1 CH2-CH3) and a HC1 variable region 1 (VH1) and the second heavy chain (HC2) of the bispecific EGFR-c-Met antibody comprises a HC2 constant domain 2 and constant domain 3 (HC2 CH2-CH3) and a HC2 variable region 2 (VH2).

In some embodiments, the bispecific antibody comprises asymmetric stabilizing mutations in the HC1 CH2-CH3 region, in the HC2 CH2-CH3 region, or both. “Asymmetric stabilizing mutations” refers to mutations in a first CH2-CH3 region and in a second CH2-CH3 region which are at different positions in the first and in the second CH2-CH3 region and favor (e.g., stabilize) heterodimer formation between the first CH2-CH3 region and the second CH2-CH3 region over homodimer formation between the first CH2-CH3 region or the second CH2-CH3 region. Exemplary asymmetric stabilizing mutations in the HC1 CH2-CH3 region and the HC2 CH2-CH3 region, or in the HC2 CH2-CH3 region and the HC1 CH2-CH3 region, are (wherein residue numbering is according to the EU Index):
F405L and K409R, respectively;
wild-type and F405L/R409K, respectively;
T366W and T366S/L368A/Y407V, respectively;
T366Y/F405A and T394W/Y407T, respectively;
T366W/F405W and T394S/Y407A, respectively;
F405W/Y407A and T366W/T394S, respectively;
L351Y/F405A/Y407V and T394W, respectively;
T366I/K392M/T394W and F405A/Y407V, respectively;
T366L/K392M/T394W and F405A/Y407V, respectively;
L351Y/Y407A and T366A/K409F, respectively;
L351Y/Y407A and T366V/K409F, respectively;
Y407A and T366A/K409F, respectively;
D399K/E356K and K409D/K392D, respectively; or
D399K/E356K/E357K and K409D/K392D/K370, respectively.

In some embodiments, the bispecific EGFR-c-Met antibody comprises an HC1 variable region comprising the amino acid sequence of SEQ ID NO:13 and a LC1 variable region comprising the amino acid sequence of SEQ ID NO:14. In some embodiments, the bispecific antibody comprises asymmetric stabilizing mutations in the HC1 CH2-CH3 region, in the HC2 CH2-CH3 region, or both. In some embodiments, the bispecific antibody comprises K409R in the c-Met binding arm and F405L in the EGFR binding arm.

In some embodiments, the bispecific EGFR-c-Met antibody comprises a HC2 variable region comprising the amino acid sequence of SEQ ID NO:15 and a LC2 variable region comprising the amino acid sequence of SEQ ID NO:16.

In some embodiments, the heavy chain 1 (HC1) comprises the amino acid sequence of SEQ ID NO: 17 and the HC2 comprises the amino acid sequence of SEQ ID NO: 19.

In some embodiments, the light chain 1 (LC1) comprises the amino acid sequence of SEQ ID NO:18 and the LC2 comprises the amino acid sequence of SEQ ID NO:20.

In some embodiments, bispecific EGFR-c-Met antibody is amivantamab or a biosimilar thereof.

Amino acid sequences of EGFR/c-Met antibody are shown in Table 1.

TABLE 1
Amino acid sequences of EGFR/c-Met antibody.

| SEQ ID NO: | Sequence |
|---|---|
| SEQ ID NO: 1 | TYGMH |
| HCDR1, EGFR binding arm | |
| (Kabat method) | |
| SEQ ID NO: 2 | VIWDDGSYKYYGDSVKG |
| HCDR2, EGFR binding arm | |
| (Kabat method) | |
| SEQ ID NO: 3 | DGITMVRGVMKDYFDY |
| HCDR3, EGFR binding arm | |
| (Kabat method) | |
| SEQ ID NO: 4 LCDR1, | RASQDISSALV |
| EGFR binding arm (Kabat | |
| method) | |
| SEQ ID NO: 5 LCDR2, | DASSLES |
| EGFR binding arm (Kabat | |
| method) | |
| SEQ ID NO: 6 LCDR3, | QQFNSYPLT |
| EGFR binding arm (Kabat | |
| method) | |
| SEQ ID NO: 7 HCDR1, c- | SYGIS |
| Met binding arm (Kabat | |
| method) | |
| SEQ ID NO: 8 HCDR2, c- | WISAYNGYTNYAQKLQG |
| Met binding arm (Kabat | |
| method) | |
| SEQ ID NO: 9 HCDR3, c- | DLRGTNYFDY |
| Met binding arm (Kabat | |
| method) | |
| SEQ ID NO: 10 LCDR1, c- | RASQGISNWLA |
| Met binding arm (Kabat | |
| method) | |
| SEQ ID NO: 11 LCDR2, c- | AASSLLS |
| Met binding arm (Kabat | |
| method) | |
| SEQ ID NO: 12 LCDR3, c- | QQANSFPIT |
| Met binding arm (Kabat | |
| method). | |
| SEQ ID NO: 13 | QVQLVESGGGVVQPGRSLRLSCAASGFTFSTYGMHWVRQAPGKG |
| VH1, EGFR binding arm | LEWVAVIWDDGSYKYYGDSVKGRFTISRDNSKNTLYL |
| | QMNSLRAEDTAVYYCARDGITMVRGVMKDYFDYWGQGTLVTVS |
| | S |
| SEQ ID NO: 14 | AIQLTQSPSSLSASVGDRVTITCRASQDISSALVWYQQKPGKAPKL |
| VL1, EGFR binding arm | IYDASSLESGVPSRFSGSESGTDFTLTISSLQPEDFATYY |
| | CQQFNSYPLTFGGGTKVEIK |
| SEQ ID NO: 15 | QVQLVQSGAEVKKPGASVKVSCETSGYTFTSYGISWVRQAPGHGI |
| VH2, c-Met binding arm | EWMGWISAYNGYTNYAQKLQGRVTMTTDTSTSTAYM |
| | ELRSLRSDDTAVYYCARDLRGTNYFDYWGQGTLVTVSS |
| SEQ ID NO: 16 | DIQMTQSPSSVSASVGDRVTITCRASQGISNWLAWFQHKPGKAPK |
| VL2, c-Met binding arm | LIYAASSLLSGVPSRFSGSGSGTDFTLTISSLOPEDFATYYCQQANSI |
| | PITFGQGTRLEIK |
| SEQ ID NO: 17 HC1 | QVQLVESGGGVVQPGRSLRLSCAASGFTFSTYGMHWVRQAPGK |
| | GLEWVAVIWDDGSYKYYGDSVKGRFTISRDNSKNTLYLQMNSL |
| | RAEDTAVYYCARDGITMVRGVMKDYFDYWGQGTLVTVSSAST |
| | KGPSVFPLAPSSKSTSGGTAALGCLVKDYFPEPVTVSWNSGALT |
| | SGVHTFPAVLQSSGLYSLSSVVTVPSSSLGTQTYICNVNHKPSNT |
| | KVDKRVEPKSCDKTHTCPPCPAPELLGGPSVFLFPPKPKDTLMIS |
| | RTPEVTCVVVDVSHEDPEVKFNWYVDGVEVHNAKTKPREEQY |
| | NSTYRVVSVLTVLHQDWLNGKEYKCKVSNKALPAPIEKTISKA |
| | KGQPREPQVYTLPPSREEMTKNQVSLTCLVKGFYPSDIAVEWES |
| | NGQPENNYKTTPPVLDSDGSFLLYSKLTVDKSRWQQGNVFSCS |
| | VMHEALHNHYTQKSLSLSPGK |
| SEQ ID NO: 18 LC1 | AIQLTQSPSSLSASVGDRVTITCRASQDISSALVWYQQKPGKAPK |
| | LLIYDASSLESGVPSRFSGSESGTDFTLTISSLQPEDFATYYCQQF |
| | NSYPLTFGGGTKVEIKRTVAAPSVFIFPPSDEQLKSGTASVVCLL |
| | NNFYPREAKVQWKVDNALQSGNSQESVTEQDSKDSTYSLSSTL |
| | TLSKADYEKHKVYACEVTHQGLSSPVTKSFNRGEC |
| SEQ ID NO: 19 HC2 | QVQLVQSGAEVKKPGASVKVSCETSGYTFTSYGISWVRQAPGH |
| | GLEWMGWISAYNGYTNYAQKLQGRVTMTTDTSTSTAYMELRS |
| | LRSDDTAVYYCARDLRGTNYFDYWGQGTLVTVSSASTKGPSVF |
| | PLAPSSKSTSGGTAALGCLVKDYFPEPVTVSWNSGALTSGVHTF |
| | PAVLQSSGLYSLSSVVTVPSSSLGTQTYICNVNHKPSNTKVDKR |
| | VEPKSCDKTHTCPPCPAPELLGGPSVFLFPPKPKDTLMISRTPEVT |
| | CVVVDVSHEDPEVKFNWYVDGVEVHNAKTKPREEQYNSTYRV |
| | VSVLTVLHQDWLNGKEYKCKVSNKALPAPIEKTISKAKGQPREP |
| | QVYTLPPSREEMTKNQVSLTCLVKGFYPSDIAVEWESNGQPENN |
| | YKTTPPVLDSDGSFFLYSRLTVDKSRWQQGNVFSCSVMHEALH |
| | NHYTQKSLSLSPGK |
| SEQ ID NO: 20 LC2 | DIQMTQSPSSVSASVGDRVTITCRASQGISNWLAWFQHKPGKAP |
| | KLLIYAASSLLSGVPSRFSGSGSGTDFTLTISSLQPEDFATYYCQQ |
| | ANSFPITFGQGTRLEIKRTVAAPSVFIFPPSDEQLKSGTASVVCLL |
| | NNFYPREAKVQWKVDNALQSGNSQESVTEQDSKDSTYSLSSTL |
| | TLSKADYEKHKVYACEVTHQGLSSPVTKSFNRGEC |

### Bispecific Antibody Formats

Exemplary antibodies of the present invention have two or more antigen binding sites. In some embodiments, the antibodies are bispecific. In some embodiments, the antibodies are multispecific.

Exemplary antibodies of the invention may be generated for example using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy-chain disulfide bonds in the hinge regions of the parent monospecific antibodies are reduced. The resulting free cysteines of one of the parent monospecific antibodies form an inter heavy-chain disulfide bond with cysteine residues of a second parent monospecific antibody molecule and simultaneously CH3 domains of the parent antibodies release and reform by dissociation-association. The CH3 domains of the Fab arms may be engineered to favor heterodimerization over homodimerization. The resulting product is a bispecific antibody having two Fab arms or half molecules which each bind a distinct epitope, i.e. an epitope on EGFR and an epitope on c-Met.

The “knob-in-hole” strategy (see, e.g., PCT Intl. Publ. No. WO 2006/028936) may be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S L368A_Y407V.

Other strategies such as promoting heavy chain heterodimerization using electrostatic interactions by substituting positively charged residues at one CH3 surface and negatively charged residues at a second CH3 surface may be used, as described in US Pat. Publ. No. US2010/0015133; US Pat. Publ. No. US2009/0182127; US Pat. Publ. No. US2010/028637 or US Pat. Publ. No. US2011/0123532. In other strategies, heterodimerization may be promoted by following substitutions (expressed as modified position in the first CH3 domain of the first heavy chain/modified position in the second CH3 domain of the second heavy chain):
L351Y_F405A_Y407V/T394W, T366I_K392M_T394W/F405A_Y407V,
T366L_K392M_T394W/F405A_Y407V, L351Y_Y407A/T366A_K409F,
L351Y_Y407A/T366V_K409F, Y407A/T366A_K409F, or
T350V_L351Y_F405A_Y407V/T350V_T366L_K392L_T394W as described in U.S. Pat. Publ. No. US2012/0149876 or U.S. Pat. Publ. No. US2013/0195849

In addition to methods described above, bispecific antibodies of the invention may be generated in vitro in a cell-free environment by introducing asymmetrical mutations in the CH3 regions of two monospecific homodimeric antibodies and forming the bispecific heterodimeric antibody from two parent monospecific homodimeric antibodies in reducing conditions to allow disulfide bond isomerization according to methods described in Intl. Pat. Publ. No. WO2011/131746. In the methods, the first monospecific bivalent antibody (e.g., anti-c-Met antibody) and the second monospecific bivalent antibody (e.g., anti-EGFR antibody) are engineered to have certain substitutions at the CH3 domain that promoter heterodimer stability; the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl)phosphine (TCEP), L-cysteine and beta-mercaptoethanol, preferably a reducing agent selected from the group consisting of: 2-mercaptoethylamine, dithiothreitol and tris(2-carboxyethyl)phosphine. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

In some embodiments, upon binding to the EGFR molecule or the c-Met molecule, the present EGFR/c-Met-binding molecule binds to the cell expressing the EGFR or c-Met protein. In some embodiments, the EGFR or c-Met-expressing cell is a cancer cell. In some embodiments, the cancer cell is selected from lung cancer, colorectal cancer, head and neck cancer, and liver cancer. In some embodiments, the cancer cell is lung cancer, colorectal cancer, head and neck cancer, or liver cancer.

### 6.4 Antibody Methodology

In some embodiments, the EGFR/c-Met binding agents described herein are anti-EGFR/c-Met antibodies. In some embodiments, the EGFR/c-Met binding agents described herein comprises one or more domain or fragments derived from an antibody (e.g., an anti-EGFR antibody or an anti-c-Met antibody). In some embodiments, the EGFR/c-Met binding agents described herein can be generated with methods and processes for the generation, selection, modification, and fragmentation, etc. of antibody molecules that are known in the art.

### 6.4.1 Antibody Fragments

The present disclosure provides antibodies and antibody fragments that bind to EGFR and/or c-Met. In certain circumstances there are advantages of using antibody fragments, rather than whole antibodies. The smaller size of the fragments allows for rapid clearance, and may lead to improved access to cells, tissues, or organs. For a review of certain antibody fragments, see Hudson et al., 2003, Nature Med. 9:129-34.

Various techniques have been developed for the production of antibody fragments. Traditionally, these fragments were derived via proteolytic digestion of intact antibodies (see, e.g., Morimoto et al., 1992, J. Biochem. Biophys. Methods 24:107-17; and Brennan et al., 1985, Science 229:81-83). However, these fragments can now be produced directly by recombinant host cells. Fab, Fv, and scFv antibody fragments can all be expressed in and secreted from E. coli or yeast cells, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., 1992, Bio/Technology 10:163-67). According to another approach, F(ab′)₂ fragments can be isolated directly from recombinant host cell culture. Fab and F(ab′)₂ fragment with increased in vivo half-life comprising salvage receptor binding epitope residues are described in, for example, U.S. Pat. No. 5,869,046. Other techniques for the production of antibody fragments will be apparent to the skilled practitioner. In certain embodiments, an antibody is a single chain Fv fragment (scFv) (see, e.g., WO 93/16185; U.S. Pat. Nos. 5,571,894 and 5,587,458). Fv and scFv have intact combining sites that are devoid of constant regions; thus, they may be suitable for reduced nonspecific binding during in vivo use. scFv fusion proteins may be constructed to yield fusion of an effector protein at either the amino or the carboxy terminus of an scFv (See, e.g., Borrebaeck ed., supra). The antibody fragment may also be a “linear antibody,” for example, as described in the references cited above. Such linear antibodies may be monospecific or multi-specific, such as bispecific.

Smaller antibody-derived binding structures are the separate variable domains (V domains) also termed single variable domain antibodies (sdAbs). Certain types of organisms, the camelids and cartilaginous fish, possess high affinity single V-like domains mounted on an Fc equivalent domain structure as part of their immune system. (Woolven et al., 1999, Immunogenetics 50:98-101; and Streltsov et al., 2004, Proc Natl Acad Sci USA. 101:12444-49). The V-like domains (called VhH in camelids and V-NAR in sharks) typically display long surface loops, which allow penetration of cavities of target antigens. They also stabilize isolated VH domains by masking hydrophobic surface patches.

These VhH and V-NAR domains have been used to engineer sdAbs. Human V domain variants have been designed using selection from phage libraries and other approaches that have resulted in stable, high binding VL- and VH-derived domains.

Antibodies provided herein include, but are not limited to, immunoglobulin molecules and immunologically active portions of immunoglobulin molecules, for example, molecules that contain an antigen binding site that bind to a EGFR or c-Met epitope. The immunoglobulin molecules provided herein can be of any class (e.g., IgG, IgE, IgM, IgD, and IgA) or any subclass (e.g., IgG1, IgG2, IgG3, IgG4, IgA1, and IgA2) of immunoglobulin molecule.

Variants and derivatives of antibodies include antibody functional fragments that retain the ability to bind to a EGFR or c-Met epitope. Exemplary functional fragments include Fab fragments (e.g., an antibody fragment that contains the antigen-binding domain and comprises a light chain and part of a heavy chain bridged by a disulfide bond); Fab′ (e.g., an antibody fragment containing a single antigen-binding domain comprising an Fab and an additional portion of the heavy chain through the hinge region); F(ab′)₂ (e.g., two Fab′ molecules joined by interchain disulfide bonds in the hinge regions of the heavy chains; the Fab′ molecules may be directed toward the same or different epitopes); a bispecific Fab (e.g., a Fab molecule having two antigen binding domains, each of which may be directed to a different epitope); a single chain comprising a variable region, also known as, scFv (e.g., the variable, antigen-binding determinative region of a single light and heavy chain of an antibody linked together by a chain of 10-25 amino acids); a disulfide-linked Fv, or dsFv (e.g., the variable, antigen-binding determinative region of a single light and heavy chain of an antibody linked together by a disulfide bond); a camelized VH (e.g., the variable, antigen-binding determinative region of a single heavy chain of an antibody in which some amino acids at the VH interface are those found in the heavy chain of naturally occurring camel antibodies); a bispecific scFv (e.g., an scFv or a dsFv molecule having two antigen-binding domains, each of which may be directed to a different epitope); a diabody (e.g., a dimerized scFv formed when the VH domain of a first scFv assembles with the VL domain of a second scFv and the VL domain of the first scFv assembles with the VH domain of the second scFv; the two antigen-binding regions of the diabody may be directed towards the same or different epitopes); and a triabody (e.g., a trimerized scFv, formed in a manner similar to a diabody, but in which three antigen-binding domains are created in a single complex; the three antigen-binding domains may be directed towards the same or different epitopes).

In some embodiments, an EGFR/c-Met binding region comprises an antigen binding fragment of an antibody. In some embodiments, the EGFR/c-Met binding region comprises a portion of an intact antibody. In some embodiments, the EGFR/c-MET binding region comprises a Fab, Fab′, F(ab′)2, Fv, single chain antibody molecules (e.g., scFv), disulfide-linked scFv (dsscFv). In some embodiments, the EGFR/c-MET binding region comprises a Fab. In other specific embodiments, an EGFR/c-MET binding region comprises two Fabs. In other specific embodiments, an EGFR/c-MET binding region comprises two Fabs in tandem. In some embodiments, the EGFR/c-MET binding region is a scFv. In some embodiments, the EGFR/c-MET binding region is selected from nanobodies, diabodies, tribodies, tetrabodies, minibodies, dual variable domain antibodies (DVD), single variable domain antibodies (e.g., camelid antibodies). Any of the VH and the VL domains described herein that bind EGFR/c-MET can be engineered into the binding molecule in the various formats described above, and their binding to EGFR/c-MET and thermostability may be assessed using the assays described herein.

In specific embodiments, an EGFR/c-MET binding region comprises an scFv that binds EGFR or c-MET. Any of the VH and the VL domains described herein that bind EGFR or c-MET may be engineered into scFv format in either VH-linker-VL or VL-linker-VH orientation. Alternatively, the VH and VL domains may be engineered into scFv format without the use of a linker in either the VH-VL or VL-VH orientation. Accordingly, in some embodiments, the EGFR/c-MET binding agent described herein comprises an scFv that binds EGFR or c-MET in the format of VH-linker-VL. In some embodiments, the EGFR/c-MET binding agent described herein comprises an scFv that binds EGFR or c-MET in the format of VL-linker-VH. In some embodiments, the EGFR or c-MET binding agent described herein comprises an scFv that binds EGFR or c-MET in the format of VH-VL. In some embodiments, the EGFR or c-MET binding agent described herein comprises an scFv that binds EGFR or c-MET in the format of VL-VH.

In recombinant expression systems, the linker is a peptide linker and may include any naturally occurring amino acid. Exemplary amino acids that can be included into the linker are Gly, Ser Pro, Thr, Glu, Lys, Arg, Ile, Leu, His and The. The linker should have a length that is adequate to link the VH and the VL in such a way that they form the correct conformation relative to one another so that they retain the desired activity, such as binding to EGFR or c-MET.

In some embodiments, the linker is about 5-50 amino acids long. In other embodiments, the linker is about 10-40 amino acids long. In other embodiments, the linker is about 10-35 amino acids long. In other embodiments, the linker is about 10-30 amino acids long. In other embodiments, the linker is about 10-25 amino acids long. In other embodiments, the linker is about 10-20 amino acids long. In other embodiments, the linker is about 15-20 amino acids long. In other embodiments, the linker is about 16-19 amino acids long. In other embodiments, the linker is 6 amino acids long. In other embodiments, the linker is 7 amino acids long. In other embodiments, the linker is 8 amino acids long. In other embodiments, the linker is 9 amino acids long. In other embodiments, the linker is 10 amino acids long. In other embodiments, the linker is 11 amino acids long. In other embodiments, the linker is 12 amino acids long. In other embodiments, the linker is 13 amino acids long. In other embodiments, the linker is 14 amino acids long. In other embodiments, the linker is 15 amino acids long. In other embodiments, the linker is 16 amino acids long. In other embodiments, the linker is 17 amino acids long. In other embodiments, the linker is 18 amino acids long. In other embodiments, the linker is 19 amino acids long. In other embodiments, the linker is 20 amino acids long. In other embodiments, the linker is 21 amino acids long. In other embodiments, the linker is 22 amino acids long. In other embodiments, the linker is 23 amino acids long. In other embodiments, the linker is 24 amino acids long. In other embodiments, the linker is 25 amino acids long. In other embodiments, the linker is 26 amino acids long. In other embodiments, the linker is 27 amino acids long. In other embodiments, the linker is 28 amino acids long. In other embodiments, the linker is 29 amino acids long. In other embodiments, the linker is 30 amino acids long. In other embodiments, the linker is 31 amino acids long. In other embodiments, the linker is 32 amino acids long. In other embodiments, the linker is 33 amino acids long. In other embodiments, the linker is 34 amino acids long. In other embodiments, the linker is 35 amino acids long. In other embodiments, the linker is 36 amino acids long. In other embodiments, the linker is 37 amino acids long. In other embodiments, the linker is 38 amino acids long. In other embodiments, the linker is 39 amino acids long. In other embodiments, the linker is 40 amino acids long. Exemplary linkers that can be used are Gly rich linkers, Gly and Ser containing linkers, Gly and Ala containing linkers, Ala and Ser containing linkers, and other flexible linkers.

Other linker sequences can include portions of immunoglobulin hinge area, CL or CH1 derived from any immunoglobulin heavy or light chain isotype. Alternatively, a variety of non-proteinaceous polymers, including polyethylene glycol (PEG), polypropylene glycol, polyoxyalkylenes, or copolymers of polyethylene glycol and polypropylene glycol, may find use as linkers. Exemplary linkers that may be used are shown in the following Table. Additional linkers are described for example in Int. Pat. Publ. No. WO2019/060695.

### 6.4.2 Fc Engineering

In some embodiments, the antigen binding domains that bind EGFR or c-Met of the disclosure are conjugated to an Ig constant region or a fragment of the Ig constant region to impart antibody-like properties, including Fc effector functions C1q binding, complement dependent cytotoxicity (CDC), Fc receptor binding, antibody-dependent cell-mediated cytotoxicity (ADCC), phagocytosis or down regulation of cell surface receptors (e.g., B cell receptor; BCR). In some embodiments, the antigen binding domains that bind EGFR or c-Met of the disclosure are used to make a fusion protein, wherein the fusion protein comprises the antigen binding domains that bind EGFR or c-Met and an Ig constant region or a fragment of the Ig constant region to impart antibody-like properties, including Fc effector functions C1q binding, complement dependent cytotoxicity (CDC), Fc receptor binding, antibody-dependent cell-mediated cytotoxicity (ADCC), trogocytosis, phagocytosis or down regulation of cell surface receptors (e.g., B cell receptor; BCR). The Ig constant region or the fragment of the Ig constant region functions also as a half-life extending moiety as discussed herein. The antigen binding domains that bind EGFR or c-Met of the disclosure may be engineered into conventional full-length antibodies using standard methods. The full-length antibodies comprising the antigen binding domain that binds EGFR or c-Met may further be engineered as described herein.

Immunoglobulin heavy chain constant region comprised of subdomains CH1, hinge, CH2 and CH3. The CH1 domain spans residues A118-V215, the CH2 domain residues A231-K340 and the CH3 domain residues G341-K447 on the heavy chain, residue numbering according to the EU Index. In some instances, G341 is referred as a CH2 domain residue. Hinge is generally defined as including E216 and terminating at P230 of human IgG1. In some embodiments, the Ig Fc region comprises at least the CH2 and the CH3 domains of the Ig constant region, and therefore comprises at least a region from about A231 to K447 of Ig heavy chain constant region.

In some embodiments, the C-terminal lysine (CTL) is removed from the Ig constant region. Accordingly, in some embodiments, the Ig Fc region comprises at least a region from about A231 to G446 of Ig heavy chain constant region. In specific embodiments, the CTL is removed from the Ig constant region by endogenous circulating carboxypeptidases in the blood stream (Cai et al., (2011) Biotechnol Bioeng 108:404-412). In some embodiments, during manufacturing, CTL removal may be controlled to less than the maximum level by control of concentration of extracellular Zn²⁺, EDTA or EDTA-Fe³⁺ as described in U.S. Patent Publ. No. US20140273092. CTL content of proteins may be measured using known methods.

In other embodiments, the antigen binding fragment that binds EGFR or c-Met fused to the Ig constant region has a C-terminal lysine content from about 10% to about 90%. In other embodiments, the C-terminal lysine content is from about 20% to about 80%. In other embodiments, the C-terminal lysine content is from about 40% to about 70%. In other embodiments, the C-terminal lysine content is from about 55% to about 70%. In other embodiments, the C-terminal lysine content is about 60%. In other embodiments, the C-terminal lysine content is less than about 20%. In other embodiments, the C-terminal lysine content is less than about 10%. In other embodiments, the C-terminal lysine content is less than about 5%. In other embodiments, the C-terminal lysine content is less than about 3%. In other embodiments, the C-terminal lysine content is less than about 2%. In other embodiments, the C-terminal lysine content is less than about 1%. In other embodiments, the C-terminal lysine content is about 0%.

The present disclosure also provides an antigen binding domain that binds EGFR or c-Met fused or conjugated to an immunoglobulin (Ig) constant region or a fragment of the Ig constant region. In some embodiments, the Ig constant region is a heavy chain constant region. In some embodiments, the Ig constant region is a light chain constant region. In some embodiments, the fragment of the Ig constant region comprises a Fc region. In some embodiments, the fragment of the Ig constant region comprises a CH2 domain. In some embodiments, the fragment of the Ig constant region comprises a CH3 domain. In some embodiments, the fragment of the Ig constant region comprises the CH2 domain and the CH3 domain. In some embodiments, the fragment of the Ig constant region comprises at least portion of a hinge, the CH2 domain and the CH3 domain. Portion of the hinge refers to one or more amino acid residues of the Ig hinge. In some embodiments, the fragment of the Ig constant region comprises the hinge, the CH2 domain and the CH3 domain.

In some embodiments, the antigen binding domain that binds EGFR or c-Met is fused or conjugated to the N-terminus of the Ig constant region or the fragment of the Ig constant region. In some embodiments, the antigen binding domain that binds EGFR or c-Met is fused or conjugated to the C-terminus of the Ig constant region or the fragment of the Ig constant region. In some embodiments, the antigen binding domain that binds EGFR or c-Met is fused or conjugated to the Ig constant region or the fragment of the Ig constant region via a second linker (L2).

The antigen binding domains that bind EGFR or c-Met of the disclosure fused or conjugated to Ig constant region or the fragment of the Ig constant region may be assessed for their functionality using several known assays. Binding to EGFR or c-Met can be assessed using methods described herein. Altered properties imparted by the Ig constant domain or the fragment of the Ig constant region such as Fc region may be assayed in Fc receptor binding assays using soluble forms of the receptors, such as the FcγRI, FcγRII, FcγRIII or FcRn receptors, or using cell-based assays measuring for example ADCC or trogocytosis.

ADCC can be assessed using an in vitro assay using EGFR or c-Met expressing cells as target cells and NK cells as effector cells. Cytolysis may be detected by the release of label (e.g. radioactive substrates, fluorescent dyes or natural intracellular proteins) from the lysed cells. In an exemplary assay, target cells are used with a ratio of 1 target cell to 4 effector cells. Target cells are pre-labeled with BATDA and combined with effector cells and the test antibody. The samples are incubated for 2 hours and cell lysis measured by measuring released BATDA into the supernatant. Data is normalized to maximal cytotoxicity with 0.67% Triton X-100 (Sigma Aldrich) and minimal control determined by spontaneous release of BATDA from target cells in the absence of any antibody.

Trogocytosis of cells may be measured for example as described in U.S. Pat. No. 11,459,391.

In some embodiments, it may be desirable to modify an anti-EGFR/c-Met antibody provided herein by Fc engineering. In certain embodiments, the modification to the Fc region of the antibody results in the decrease or elimination of an effector function of the antibody. In certain embodiments, the effector function is ADCC, ADCP, and/or CDC. In some embodiments, the effector function is ADCC. In other embodiments, the effector function is ADCP. In other embodiments, the effector function is CDC. In one embodiment, the effector function is ADCC and ADCP. In one embodiment, the effector function is ADCC and CDC. In one embodiment, the effector function is ADCP and CDC. In one embodiment, the effector function is ADCC, ADCP and CDC. This may be achieved by introducing one or more amino acid substitutions in an Fc region of the antibody. For example, substitutions into human IgG1 using IgG2 residues at positions 233-236 and IgG4 residues at positions 327, 330, and 331 were shown to greatly reduce ADCC and CDC (see, e.g., Armour et al., 1999, Eur. J. Immunol. 29 (8): 2613-24; and Shields et al., 2001, J. Biol. Chem. 276 (9): 6591-604). Other Fc variants are provided elsewhere herein.

To increase the serum half-life of the antibody, one may incorporate a salvage receptor binding epitope into the antibody (especially an antibody fragment), for example, as described in U.S. Pat. No. 5,739,277. Term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

In some embodiments, the Ig constant region or the fragment of the Ig constant region comprises at least one mutation that modulates a half-life of the EGFR/c-Met binding agent. In some embodiments, the at least one mutation that modulates the half-life of the MLSN binding agent is selected from the group consisting of H435A, P257I/N434H, D376V/N434H, M252Y/S254T/T256E/H433K/N434F, T308P/N434A, and H435R, wherein residue numbering is according to the EU index. In some embodiments, the EGFR/c-Met binding agent comprises a first Ig constant region or a fragment thereof and a second Ig constant region or a fragment thereof. In some embodiments, one or both of the first Ig constant region or a fragment thereof and a second Ig constant region or a fragment thereof comprises at least one mutation that modulates a half-life of the EGFR/c-Met binding agent independently selected from the group consisting of H435A, P257I/N434H, D376V/N434H, M252Y/S254T/T256E/H433K/N434F, T308P/N434A, and H435R, wherein residue numbering is according to the EU index.

In some embodiments, the Ig constant region or the fragment of the Ig constant region comprises at least one mutation that results in reduced binding of the EGFR/c-Met binding agent to a FcγR.

In some embodiments, the at least one mutation that results in reduced binding of the EGFR or c-Met binding agent to the FcγR is selected from the group consisting of F234A/L235A, L234A/L235A, L234A/L235A/D265S, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index. In some embodiments, the EGFR/c-Met binding agent comprises a first Ig constant region or a fragment thereof and a second Ig constant region or a fragment thereof. In some embodiments, one or both of the first Ig constant region or a fragment thereof and a second Ig constant region or a fragment thereof comprises at least one mutation that modulates a half-life of the MLSN binding agent independently selected from the group consisting of F234A/L235A, L234A/L235A, L234A/L235A/D265S, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index.

In some embodiments, the Ig constant region or the fragment of the Ig constant region comprises at least one mutation that results in enhanced binding of the EGFR/c-Met binding agent to a FcγR.

In some embodiments, the at least one mutation that results in enhanced binding of the EGFR/c-Met binding agent to the FcγR is selected from the group consisting of S239D/I332E, S298A/E333A/K334A, F243L/R292P/Y300L, F243L/R292P/Y300L/P396L, F243L/R292P/Y300L/V305I/P396L and G236A/S239D/I332E, wherein residue numbering is according to the EU index. In some embodiments, the FcγR is FcγRI, FcγRIIA, FcγRIIB or FcγRIII, or any combination thereof. In some embodiments, the EGFR or c-Met binding agent comprises a first Ig constant region or a fragment thereof and a second Ig constant region or a fragment thereof. In some embodiments, one or both of the first Ig constant region or a fragment thereof and a second Ig constant region or a fragment thereof comprises at least one mutation that modulates a half-life of the MLSN binding agent independently selected from the group consisting of S239D/I332E, S298A/E333A/K334A, F243L/R292P/Y300L, F243L/R292P/Y300L/P396L, F243L/R292P/Y300L/V305I/P396L and G236A/S239D/I332E, wherein residue numbering is according to the EU index.

In some embodiments, the EGFR/c-Met binding agent comprises at least one mutation in a CH3 domain of a first Ig constant region or in a CH3 domain of the fragment of the first Ig constant region and/or at least one mutation in a CH3 domain of a second Ig constant region or in a CH3 domain of the fragment of the second Ig constant region. In some embodiments, the at least one mutation in a CH3 domain of the first Ig constant region or in a CH3 domain of the fragment of the first Ig constant region and/or at least one mutation in a CH3 domain of the second Ig constant region or in a CH3 domain of the fragment of the second Ig constant region is selected from the group consisting of T350V, L351Y, F405A, Y407V, T366Y, T366W, T366L, F405W, K392L, T394W, T394S, Y407T, Y407A, H435R, Y436F, T366S/L368A/Y407V, L351Y/F405A/Y407V, T366I/K392M/T394W, T366L/K392L/T394W, F405A/Y407V, T366L/K392M/T394W, L351Y/Y407A, L351Y/Y407V, T366A/K409F, L351Y/Y407A, T366V/K409F, T366A/K409F, T350V/L351Y/F405A/Y407V, T350V/T366L/K392L/T394W, and H435R/L436F wherein residue numbering is according to the EU index. In some embodiments, at least one mutation in the CH3 domain is selected from the group consisting of H435R, Y436F and H435R/L436F, wherein residue numbering is according to the EU index

In some embodiments, the first Ig constant region or the fragment of the first Ig constant region and the second Ig constant region or the fragment of the second Ig constant region comprise the following mutations
L234A_L235A_D265S_T350V_L351Y_F405A_Y407V in the first Ig constant region and
L234A_L235A_D265S_T350V_T366L_K392L_T394W in the second Ig constant region; or
L234A_L235A_D265S_T350V_T366L_K392L_T394W in the first Ig constant region and
L234A_L235A_D265S_T350V_L351Y_F405A_Y407V in the second Ig constant region.

### 6.5 Variant Bispecific EGFR/c-Met Binding Agents and Compositions

In another general aspect, the disclosure relates to an isolated variant bispecific EGFR/c-Met binding agent or antigen binding region thereof provided herein.

In one aspect, provided herein is a composition comprising a variant EGFR/c-Met binding agent comprising a first polypeptide comprising SEQ ID NO: 17, a second polypeptide comprising SEQ ID NO: 18, a third polypeptide comprising SEQ ID NO: 19, and a fourth polypeptide comprising SEQ ID NO: 20, wherein Asp99 of SEQ ID NO: 17 is isomerized, wherein the residue numbering starts from N-terminus of SEQ ID NO: 17. In some embodiments, the composition further comprises a non-modified EGFR/c-Met binding agent comprising a first polypeptide comprising SEQ ID NO: 17, a second polypeptide comprising SEQ ID NO: 18, a third polypeptide comprising SEQ ID NO: 19, and a fourth polypeptide comprising SEQ ID NO: 20.

In another aspect, provided herein is a composition comprising a variant EGFR/c-Met binding agent comprising the VH and VL sequences, respectively, of SEQ ID NO: 17, SEQ ID NO: 18, SEQ ID NO: 19, and SEQ ID NO: 20, wherein Asp99 of SEQ ID NO: 17 is isomerized, wherein the residue numbering starts from N-terminus of SEQ ID NO: 17. In some embodiments, the variant EGFR/c-Met binding agent comprises the VH of SEQ ID NO: 13, wherein Asp99 of SEQ ID NO: 13 is isomerized, wherein the residue numbering starts from N-terminus of SEQ ID NO: 13.

In some embodiments, the composition comprising the variant EGFR/c-Met binding agent and the non-modified EGFR/c-Met binding agent disclosed herein, comprises ≤about 10% of isomerized Asp99 variant. In some embodiments, the composition comprising the variant EGFR/c-Met binding agent and the non-modified EGFR/c-Met binding agent disclosed herein, comprises ≤about 9% of isomerized Asp99 variant. In some embodiments, the composition comprising the variant EGFR/c-Met binding agent and the non-modified EGFR/c-Met binding agent disclosed herein, comprises ≤about 8% of isomerized Asp99 variant. In some embodiments, the composition comprising the variant EGFR/c-Met binding agent and the non-modified EGFR/c-Met binding agent disclosed herein, comprises ≤about 7% of isomerized Asp99 variant. In some embodiments, the composition comprising the variant EGFR/c-Met binding agent and the non-modified EGFR/c-Met binding agent disclosed herein, comprises ≤about 6% of isomerized Asp99 variant. In some embodiments, the composition comprising the variant EGFR/c-Met binding agent and the non-modified EGFR/c-Met binding agent disclosed herein, comprises ≤about 5% of isomerized Asp99 variant. In some embodiments, the composition comprising the variant EGFR/c-Met binding agent and the non-modified EGFR/c-Met binding agent disclosed herein, comprises ≤about 4% of isomerized Asp99 variant. In some embodiments, the composition comprising the variant EGFR/c-Met binding agent and the non-modified EGFR/c-Met binding agent disclosed herein, comprises ≤about 3% of isomerized Asp99 variant. In some embodiments, the composition comprising the variant EGFR/c-Met binding agent and the non-modified EGFR/c-Met binding agent disclosed herein, comprises ≤about 2% of isomerized Asp99 variant. In some embodiments, the composition comprising the variant EGFR/c-Met binding agent and the non-modified EGFR/c-Met binding agent disclosed herein, comprises ≤about 1.5% of isomerized Asp99 variant. In some embodiments, the foregoing compositions comprise the isomerized Asp99 variant such that the amount is measurable and greater than zero.

In some embodiments, the composition comprising the variant EGFR/c-Met binding agent and the non-modified EGFR/c-Met binding agent disclosed herein, comprises from more than about 0% to about 10% of isomerized Asp99 variant. In some embodiments, the composition comprising the variant EGFR/c-Met binding agent and the non-modified EGFR/c-Met binding agent disclosed herein, comprises from about 0.1% to about 10% of isomerized Asp99 variant. In some embodiments, the composition comprising the variant EGFR/c-Met binding agent and the non-modified EGFR/c-Met binding agent disclosed herein, comprises from about 0.3% to about 10% of isomerized Asp99 variant. In some embodiments, the composition comprising the variant EGFR/c-Met binding agent and the non-modified EGFR/c-Met binding agent disclosed herein, comprises from about 0.5% to about 10% of isomerized Asp99 variant. In some embodiments, the composition comprising the variant EGFR/c-Met binding agent and the non-modified EGFR/c-Met binding agent disclosed herein, comprises from about 0.7% to about 10% of isomerized Asp99 variant. In some embodiments, the composition comprising the variant EGFR/c-Met binding agent and the non-modified EGFR/c-Met binding agent disclosed herein, comprises from about 0.8% to about 10% of isomerized Asp99 variant. In some embodiments, the composition comprising the variant EGFR/c-Met binding agent and the non-modified EGFR/c-Met binding agent disclosed herein, comprises from about 0.9% to about 10% of isomerized Asp99 variant. In some embodiments, the composition comprising the variant EGFR/c-Met binding agent and the non-modified EGFR/c-Met binding agent disclosed herein, comprises from about 1% to about 10% of isomerized Asp99 variant. In some embodiments, the composition comprising the variant EGFR/c-Met binding agent and the non-modified EGFR/c-Met binding agent disclosed herein, comprises from about 1.5% to about 10% of isomerized Asp99 variant. In some embodiments, the composition comprising the variant EGFR/c-Met binding agent and the non-modified EGFR/c-Met binding agent disclosed herein, comprises from about 2% to about 10% of isomerized Asp99 variant. In some embodiments, the composition comprising the variant EGFR/c-Met binding agent and the non-modified EGFR/c-Met binding agent disclosed herein, comprises from about 2.5% to about 10% of isomerized Asp99 variant. In some embodiments, the composition comprising the variant EGFR/c-Met binding agent and the non-modified EGFR/c-Met binding agent disclosed herein, comprises from about 3% to about 10% of isomerized Asp99 variant. In some embodiments, the composition comprising the variant EGFR/c-Met binding agent and the non-modified EGFR/c-Met binding agent disclosed herein, comprises from about 3.5% to about 10% of isomerized Asp99 variant. In some embodiments, the composition comprising the variant EGFR/c-Met binding agent and the non-modified EGFR/c-Met binding agent disclosed herein, comprises from about 4% to about 10% of isomerized Asp99 variant. In some embodiments, the composition comprising the variant EGFR/c-Met binding agent and the non-modified EGFR/c-Met binding agent disclosed herein, comprises from about 4.5% to about 10% of isomerized Asp99 variant. In some embodiments, the composition comprising the variant EGFR/c-Met binding agent and the non-modified EGFR/c-Met binding agent disclosed herein, comprises from about 5% to about 10% of isomerized Asp99 variant. In some embodiments, the composition comprising the variant EGFR/c-Met binding agent and the non-modified EGFR/c-Met binding agent disclosed herein, comprises from about 5.5% to about 10% of isomerized Asp99 variant. In some embodiments, the composition comprising the variant EGFR/c-Met binding agent and the non-modified EGFR/c-Met binding agent disclosed herein, comprises from about 6% to about 10% of isomerized Asp99 variant. In some embodiments, the composition comprising the variant EGFR/c-Met binding agent and the non-modified EGFR/c-Met binding agent disclosed herein, comprises from about 6.5% to about 10% of isomerized Asp99 variant. In some embodiments, the composition comprising the variant EGFR/c-Met binding agent and the non-modified EGFR/c-Met binding agent disclosed herein, comprises from about 7% to about 10% of isomerized Asp99 variant. In some embodiments, the composition comprising the variant EGFR/c-Met binding agent and the non-modified EGFR/c-Met binding agent disclosed herein, comprises from about 7.5% to about 10% of isomerized Asp99 variant. In some embodiments, the composition comprising the variant EGFR/c-Met binding agent and the non-modified EGFR/c-Met binding agent disclosed herein, comprises from about 8% to about 10% of isomerized Asp99 variant. In some embodiments, the composition comprising the variant EGFR/c-Met binding agent and the non-modified EGFR/c-Met binding agent disclosed herein, comprises from about 8.5% to about 10% of isomerized Asp99 variant. In some embodiments, the composition comprising the variant EGFR/c-Met binding agent and the non-modified EGFR/c-Met binding agent disclosed herein, comprises from about 9% to about 10% of isomerized Asp99 variant. In some embodiments, the composition comprising the variant EGFR/c-Met binding agent and the non-modified EGFR/c-Met binding agent disclosed herein, comprises from about 9.5% to about 10% of isomerized Asp99 variant. In some embodiments, the foregoing compositions comprise the isomerized Asp99 variant such that the amount is measurable and greater than zero.

In some embodiments, the composition facilitates binding to any one or more of EGFR and c-Met.

In some embodiments described herein, the bispecific EGFR/c-Met antibody of the invention has a biantennary glycan structure with fucose content of about between 0% to about 20%, for example about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, or any amount in between. In some embodiments, the bispecific EGFR/c-Met antibody of the invention has a biantennary glycan structure with fucose content of about between 0.5% to about 20%. In some embodiments, the bispecific EGFR/c-Met antibody of the invention has a biantennary glycan structure with fucose content of about between 1% to about 19%. In some embodiments, the bispecific EGFR/c-Met antibody of the invention has a biantennary glycan structure with fucose content of about between 1% to about 18%. In some embodiments, the bispecific EGFR/c-Met antibody of the invention has a biantennary glycan structure with fucose content of about between 1% to about 17%. In some embodiments, the bispecific EGFR/c-Met antibody of the invention has a biantennary glycan structure with fucose content of about between 1% to about 16%. In some embodiments, the bispecific EGFR/c-Met antibody of the invention has a biantennary glycan structure with fucose content of about between 1% to about 15%. In some embodiments, the bispecific EGFR/c-Met antibody of the invention has a biantennary glycan structure with fucose content of about between 2% to about 17%. In some embodiments, the bispecific EGFR/c-Met antibody of the invention has a biantennary glycan structure with fucose content of about between 3% to about 17%. In some embodiments, the bispecific EGFR/c-Met antibody of the invention has a biantennary glycan structure with fucose content of about between 4% to about 17%. In some embodiments, the bispecific EGFR/c-Met antibody of the invention has a biantennary glycan structure with fucose content of about between 5% to about 17%. In some embodiments, the bispecific EGFR/c-Met antibody of the invention has a biantennary glycan structure with fucose content of about between 6% to about 17%. In some embodiments, the bispecific EGFR/c-Met antibody of the invention has a biantennary glycan structure with fucose content of about between 7% to about 17%. In some embodiments, the bispecific EGFR/c-Met antibody of the invention has a biantennary glycan structure with fucose content of about between 8% to about 17%.

In some embodiments, the composition comprising the variant EGFR/c-Met binding agent comprising a first polypeptide comprising SEQ ID NO: 17, a second polypeptide comprising SEQ ID NO: 18, a third polypeptide comprising SEQ ID NO: 19, and a fourth polypeptide comprising SEQ ID NO: 20, wherein Asp99 of SEQ ID NO: 17 is isomerized, wherein the variant EGFR/c-Met binding agent has a biantennary glycan structure with fucose content of about between 0% to about 20%, for example about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, or any amount in between. In some embodiments, the composition further comprises a non-modified EGFR/c-Met binding agent comprising a first polypeptide comprising SEQ ID NO: 17, a second polypeptide comprising SEQ ID NO: 18, a third polypeptide comprising SEQ ID NO: 19, and a fourth polypeptide comprising SEQ ID NO: 20, wherein the variant EGFR/c-Met binding agent has a biantennary glycan structure with fucose content of about between 0% to about 20%, for example about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, or any amount in between.

In some embodiments, the composition comprising the non-modified EGFR/c-Met binding agent comprising a first polypeptide comprising SEQ ID NO: 17, a second polypeptide comprising SEQ ID NO: 18, a third polypeptide comprising SEQ ID NO: 19, and a fourth polypeptide comprising SEQ ID NO: 20 wherein the the variant EGFR/c-Met binding agent has a biantennary glycan structure with fucose content of about between 0% to about 20%, for example about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, or any amount in between.

In some embodiments, the composition comprising the variant EGFR/c-Met binding agent comprising a first polypeptide comprising SEQ ID NO: 17, a second polypeptide comprising SEQ ID NO: 18, a third polypeptide comprising SEQ ID NO: 19, and a fourth polypeptide comprising SEQ ID NO: 20, wherein Asp99 of SEQ ID NO: 17 is isomerized, has an impact on EGFR bioactivity. In some embodiments, the bioactivity is ADCC of the EGFR/c-Met bispecific antibodies.

The EGFR bioactivity can be measured using assays known in the art. In some embodiments, the EGFR bioactivity can be measured using a cell-based luciferase reporter assay kit targeting EGFR, wherein the luciferase reporter gene functionally linked to a EGFR-responsive promoter. In some embodiments, the EGFR bioactivity of the composition comprising the variant EGFR/c-Met binding agent is calculated relative to the EGFR/c-Met bispecific antibodies reference material (RM).

The ADCC of the EGFR/c-Met bispecific antibodies can be measured using assays known in the art. In some embodiments, the ADCC can be measured using quantifying the lysis of EGFR-expressing target cells by effector cells in the presence of the EGFR/c-Met bispecific antibodies, such as the 51Cr release assay, LDH release assay, or live-cell imaging assays. In some embodiments, the effector cells are T lymphocyte cells or T cells. In some embodiments, the T cells are Jurkat cells. In some embodiments, the Jurkat cells are engineered to express the FcγRIIIa receptor. In some embodiments, the ADCC of the composition comprising the variant EGFR/c-Met binding agent is calculated relative to the EGFR/c-Met bispecific antibodies reference material (RM).

### 6.5.1 Compositions Comprising EGFR/c-Met Binding Agents and Hyaluronidase Enzyme.

In some embodiments, the composition can comprise an amount of hyaluronidase enzyme that results in an increase in the dispersion of the bispecific antibody during the subcutaneous administration. The hyaluronidase enzyme has no adverse effect on the molecular integrity of the bispecific antibody. Furthermore, the hyaluronidase enzyme merely modifies the delivery of the bispecific antibody to the systemic circulation but does not possess any properties that could provide or contribute to the therapeutic effects of systemically absorbed bispecific antibody. The hyaluronidase enzyme is not systemically bioavailable and does not adversely affect the molecular integrity of the bispecific antibody at the recommended storage conditions of the composition. A number of suitable hyaluronidase enzymes are known. The preferred enzyme is a human hyaluronidase enzyme, such as a soluble human PH20 hyaluronidase, preferably the recombinant human hyaluronidase enzyme product known as rHuPH20. The amino acid sequence of soluble human PH20 hyaluronidases include the soluble human PH20 known as rHuPH20 and available under CAS Registry No. 757971-58-7. Soluble human PH20 hyaluronidases are described in Int'l Pub. No. WO2004/078140 and U.S. Pat. No. 7,767,429 incorporated herein by reference, in their entirety. In some embodiments, soluble hyaluronidases include those whose sequence are set forth in any of SEQ ID NOs: 21-25. Soluble PH20 hyaluronidase, when expressed in a cell, include a signal sequence for trafficking in the cell. Thus, in some embodiments, the amino acid sequence of the soluble PH20 hyaluronidase comprises SEQ ID NO: 26. In some embodiments, the amino acid sequence of the soluble PH20 hyaluronidase comprises SEQ ID NO: 22, namely residues 36-482 of wild type human hyaluronidase. In some embodiments, the amino acid sequence of the soluble PH20 hyaluronidase comprises SEQ ID NO: 23. In some embodiments, the amino acid sequence of the soluble PH20 hyaluronidase comprises SEQ ID NO: 24. In some embodiments, the soluble PH20 hyaluronidase comprises rHuPH20 comprising the amino acid sequence of SEQ ID NO: 25. In some embodiments, the amino acid sequence of the soluble PH20 hyaluronidase comprises SEQ ID NO: 21. In some embodiments, the soluble PH20 hyaluronidases, when expressed in a cell, comprise a mixture of species that can include any one of SEQ ID NOs: 21, 22, 23, 24, and 25 in various abundance. The average molecular weight is 61 kDa.

rHuPH20 refers to the composition produced upon expression in a cell, such as CHO cell, of nucleic acids that encode residues 36-482 of SEQ ID NO: 26, generally linked to the native or a heterologous signal sequence (residues 1-35 of SEQ ID NO: 26). rHuPH20 is produced by expression of a nucleic acid molecule, such as encoding amino acids 1-482 (set forth in SEQ ID NO: 26) in a mammalian cell. Translational processing removes the 35 amino acid signal sequence. As produced in the culture medium there is heterogeneity at the C-terminus such that the product, designated rHuPH20, includes a mixture of species that can include one or more of the polypeptides 36-480, 36-481, and 36-482 of SEQ ID NO: 26, and some shorter polypeptides, in various abundance. Typically, rHuPH20 is produced in cells, such as CHO cells, for example DG44 CHO cells, that facilitate correct N-glycosylation to retain activity. In some embodiments, the hyaluronidase is a soluble human PH20 (rHuPH20) comprising the amino acid sequence of SEQ ID NO: 25. In some embodiments, the hyaluronidase is a soluble human PH20 (rHuPH20) comprising the amino acid sequence of SEQ ID NO: 21. In some embodiments, the hyaluronidase is a soluble human PH20 (rHuPH20) comprising the amino acid sequence of SEQ ID NO: 22. In some embodiments, the hyaluronidase is a soluble human PH20 (rHuPH20) comprising the amino acid sequence of SEQ ID NO: 23. In some embodiments, the hyaluronidase is a soluble human PH20 (rHuPH20) comprising the amino acid sequence of SEQ ID NO: 24.

TABLE 2
Amino acid sequences of rHuPH20 variants.

| | |
|---|---|
| SEQ ID NO: 21: | LNFRAPPVIPNVPELWAWNAPSEFCLGKFDEPLDMSLESFIGS |
| rHuPH20 | PRINATGQGVTIFYVDRLGYYPYIDSITGVTVNGGIPQKISLQ |
| | DHLDKAKKDITFYMPVDNLGMAVIDWEEWRPTWARNWKPKDVY |
| | KNRSIELVQQQNVQLSLTEATEKAKQEFEKAGKDELVETIKLG |
| | KLLRPNHLWGYYLFPDCYNHHYKKPGYNGSCFNVEIKRNDDLS |
| | WLWNESTALYPSIYLNTQQSPVAATLYVRNRVREAIRVSKIPD |
| | AKSPLPVFAYTRIVFTDQVLKFLSQDELVYTFGETVALGASGI |
| | VIWGTLSIMRSMKSCLLLDNYMETILNPYIINVTLAAKMCSQV |
| | LCQEQGVCIRKNWNSSDYLHLNPDNFAIQLEKGGKFTVRGKPT |
| | LEDLEQFSEKFYCSCYSTLSCKEKADVKDTDAVDVCIADGVCI |
| | DAFLKPPMETEEP |
| SEQ ID NO: 22: | LNFRAPPVIPNVPELWAWNAPSEFCLGKFDEPLDMSLFSFIGS |
| rHuPH20 variant 1 | PRINATGQGVTIFYVDRLGYYPYIDSITGVTVNGGIPQKISLQ |
| | DHLDKAKKDITFYMPVDNLGMAVIDWEEWRPTWARNWKPKDVY |
| | KNRSIELVQQQNVQLSLTEATEKAKQEFEKAGKDFLVETIKLG |
| | KLLRPNHLWGYYLFPDCYNHHYKKPGYNGSCFNVEIKRNDDLS |
| | WLWNESTALYPSIYLNTQQSPVAATLYVRNRVREAIRVSKIPD |
| | AKSPLPVFAYTRIVFTDQVLKELSQDELVYTFGETVALGASGI |
| | VIWGTLSIMRSMKSCLLLDNYMETILNPYIINVTLAAKMCSQV |
| | LCQEQGVCIRKNWNSSDYLHLNPDNFAIQLEKGGKFTVRGKPT |
| | LEDLEQFSEKFYCSCYSTLSCKEKADVKDTDAVDVCIADGVCI |
| | DAFLKPPMETEEPQIFY |
| SEQ ID NO: 23: | LNFRAPPVIPNVPFLWAWNAPSEFCLGKFDEPLDMSLESFIGS |
| rHuPH20 variant 2 | PRINATGQGVTIFYVDRLGYYPYIDSITGVTVNGGIPQKISLQ |
| | DHLDKAKKDITFYMPVDNLGMAVIDWEEWRPTWARNWKPKDVY |
| | KNRSIELVQQQNVQLSLTEATEKAKQEFEKAGKDFLVETIKLG |
| | KLLRPNHLWGYYLFPDCYNHHYKKPGYNGSCFNVEIKRNDDLS |
| | WLWNESTALYPSIYLNTQQSPVAATLYVRNRVREAIRVSKIPD |
| | AKSPLPVFAYTRIVFTDQVLKFLSQDELVYTFGETVALGASGI |
| | VIWGTLSIMRSMKSCLLLDNYMETILNPYIINVTLAAKMCSQV |
| | LCQEQGVCIRKNWNSSDYLHLNPDNFAIQLEKGGKFTVRGKPT |
| | LEDLEQFSEKFYCSCYSTLSCKEKADVKDTDAVDVCIADGVCI |
| | DAFLKPPMETEEPQIF |
| SEQ ID NO: 24: | LNFRAPPVIPNVPFLWAWNAPSEFCLGKFDEPLDMSLESFIGS |
| rHuPH20 variant 3 | PRINATGQGVTIFYVDRLGYYPYIDSITGVTVNGGIPQKISLQ |
| | DHLDKAKKDITFYMPVDNLGMAVIDWEEWRPTWARNWKPKDVY |
| | KNRSIELVQQQNVQLSLTEATEKAKQEFEKAGKDFLVETIKLG |
| | KLLRPNHLWGYYLFPDCYNHHYKKPGYNGSCFNVEIKRNDDLS |
| | WLWNESTALYPSIYLNTQQSPVAATLYVRNRVREAIRVSKIPD |
| | AKSPLPVFAYTRIVFTDQVLKELSQDELVYTEGETVALGASGI |
| | VIWGTLSIMRSMKSCLLLDNYMETILNPYIINVTLAAKMCSQV |
| | LCQEQGVCIRKNWNSSDYLHLNPDNFAIQLEKGGKFTVRGKPT |
| | LEDLEQFSEKFYCSCYSTLSCKEKADVKDTDAVDVCIADGVCI |
| | DAFLKPPMETEEPQI |
| SEQ ID NO: 25: | LNFRAPPVIPNVPELWAWNAPSEFCLGKFDEPLDMSLESFIGS |
| rHuPH20 variant 4 | PRINATGQGVTIFYVDRLGYYPYIDSITGVTVNGGIPQKISLQ |
| | DHLDKAKKDITFYMPVDNLGMAVIDWEEWRPTWARNWKPKDVY |
| | KNRSIELVQQQNVQLSLTEATEKAKQEFEKAGKDFLVETIKLG |
| | KLLRPNHLWGYYLFPDCYNHHYKKPGYNGSCFNVEIKRNDDLS |
| | WLWNESTALYPSIYLNTQQSPVAATLYVRNRVREAIRVSKIPD |
| | AKSPLPVFAYTRIVFTDQVLKELSQDELVYTFGETVALGASGI |
| | VIWGTLSIMRSMKSCLLLDNYMETILNPYIINVTLAAKMCSQV |
| | LCQEQGVCIRKNWNSSDYLHLNPDNFAIQLEKGGKFTVRGKPT |
| | LEDLEQFSEKFYCSCYSTLSCKEKADVKDTDAVDVCIADGVCI |
| | DAFLKPPMETEEPQ |
| SEQ ID NO: 26 | MGVLKFKHIFFRSFVKSSGVSQIVFTFLLIPCCLTLNFRAPPV |
| | IPNVPFLWAWNAPSEFCLGKFDEPLDMSLESFIGSPRINATGQ |
| | GVTIFYVDRLGYYPYIDSITGVTVNGGIPQKISLQDHLDKAKK |
| | DITFYMPVDNLGMAVIDWEEWRPTWARNWKPKDVYKNRSIELV |
| | QQQNVQLSLTEATEKAKQEFEKAGKDFLVETIKLGKLLRPNHL |
| | WGYYLFPDCYNHHYKKPGYNGSCFNVEIKRNDDLSWLWNESTA |
| | LYPSIYLNTQQSPVAATLYVRNRVREAIRVSKIPDAKSPLPVF |
| | AYTRIVFTDQVLKFLSQDELVYTFGETVALGASGIVIWGTLSI |
| | MRSMKSCLLLDNYMETILNPYIINVTLAAKMCSQVLCQEQGVC |
| | IRKNWNSSDYLHLNPDNFAIQLEKGGKFTVRGKPTLEDLEQFS |
| | EKFYCSCYSTLSCKEKADVKDTDAVDVCIADGVCIDAFLKPPM |
| | ETEEPQIFYNASPSTLSATMFIVSILFLIISSVASL |
| SEQ ID NO: 27 | LNFRAPPVIPNVPELWAWNAPSEFCLGKFDEPLDMSLESFIGS |
| | PRINATGQGVTIFYVDRLGYYPYIDSITGVTVNGGIPQKISLQ |
| | DHLDKAKKDITFYMPVDNLGMAVIDWEEWRPTWARNWKPKDVY |
| | KNRSIELVQQQNVQLSLTEATEKAKQEFEKAGKDFLVETIKLG |
| | KLLRPNHLWGYYLFPDCYNHHYKKPGYNGSCFNVEIKRNDDLS |
| | WLWNESTALYPSIYLNTQQSPVAATLYVRNRVREAIRVSKIPD |
| | AKSPLPVFAYTRIVFTDQVLKELSQDELVYTFGETVALGASGI |
| | VIWGTLSIMRSMKSCLLLDNYMETILNPYIINVTLAAKMCSQV |
| | LCQEQGVCIRKNWNSSDYLHLNPDNFAIQLEKGGKFTVRGKPT |
| | LEDLEQFSEKFYCSCYSTLSCKEKADVKDTDAVDVCIADGVCI |
| | DAFLKPPMETEEPQIFYNASPSTLSATMFIVSILFLIISSVAS |
| | L |
| SEQ ID NO: 28 | MGVLKFKHIFFRSFVKSSGVSQIVFTELLIPCCLTLNFRAPPV |
| | IPNVAFLWAWNAPSEFCLGKFDEPLDMSLESFIGSPRINATGQ |
| | GVTIFYVDRLGYYPYIDSITGVTVNGGIPQKISLQDHLDKAKK |
| | DITFYMPVDNLGMAVIDWEEWRPTWARNWKPKDVYKNRSIELV |
| | QQQNVQLSLTEATEKAKQEFEKAGKDFLVETIKLGKLLRPNHL |
| | WGYYLFPDCYNHHYKKPGYNGSCFNVEIKRNDDLSWLWNESTA |
| | LYPSIYLNTQQSPVAATLYVRNRVREAIRVSKIPDAKSPLPVF |
| | AYTRIVFTDQVLKELSQDELVYTFGETVALGASGIVIWGTLSI |
| | MRSMKSCLLLDNYMETILNPYIINVTLAAKMCSQVLCQEQGVC |
| | IRKNWNSSDYLHLNPDNFAIQLEKGGKFTVRGKPTLEDLEQES |
| | EKFYCSCYSTLSCKEKADVKDTDAVDVCIADGVCIDAFLKPPM |
| | ETEEPQIFYNASPSTLSATMFIVSIWFLIISSVASL |
| SEQ ID NO: 29 | LNFRAPPVIPNVAFLWAWNAPSEFCLGKFDEPLDMSLESFIGS |
| | PRINATGQGVTIFYVDRLGYYPYIDSITGVTVNGGIPQKISLQ |
| | DHLDKAKKDITFYMPVDNLGMAVIDWEEWRPTWARNWKPKDVY |
| | KNRSIELVQQQNVQLSLTEATEKAKQEFEKAGKDFLVETIKLG |
| | KLLRPNHLWGYYLFPDCYNHHYKKPGYNGSCFNVEIKRNDDLS |
| | WLWNESTALYPSIYLNTQQSPVAATLYVRNRVREAIRVSKIPD |
| | AKSPLPVFAYTRIVFTDQVLKFLSQDELVYTFGETVALGASGI |
| | VIWGTLSIMRSMKSCLLLDNYMETILNPYIINVTLAAKMCSQV |
| | LCQEQGVCIRKNWNSSDYLHLNPDNFAIQLEKGGKFTVRGKPT |
| | LEDLEQFSEKFYCSCYSTLSCKEKADVKDTDAVDVCIADGVCI |
| | DAFLKPPMETEEPQIFYNASPSTLSATMFIVSIWFLIISSVAS |
| | L |
| SEQ ID NO: 30 | MGVLQFKHIFFRSFVKSSGVSQIVFTFLLIPCCLTLNFRAPPV |
| | IPNVPELWAWNAPSEFCLGKFDEPLDMSLESFIGSPRINATGQ |
| | GVTIFYVDRLGYYPYIDSITGVTVNGGIPQKISLQDHLDKAKK |
| | DITFYMPVDNLGMAVIDWEEWRPTWARNWKPKDVYKNRSIELV |
| | QQQNVQLSLTEATEKAKQEFEKAGKDFLVETIKLGKLLRPNHL |
| | WGYYLFPDCYNHHYKKPGYNGSCFNVEIKRNDDLSWLWNESTA |
| | LYPSIYLNTQQSPVAATLYVRNRVREAIRVSKIPDAKSPLPVF |
| | AYTRIVFTDQVLKFLSQDELVYTFGETVALGASGIVIWGTLSI |
| | MRSMKSCLLLDNYMETILNPYIINVTLAAKMCSQVLCQEQGVC |
| | IRKNWNSSDYLHLNPDNFAIQLEKGGKFTVRGKPTLEDLEQES |
| | EKFYCSCYSTLSCKEKADVKDTDAVDVCIADGVCIDAFLKPPM |
| | ETEEPQIFYNASPSTLSATMFIVSILFLIISSVASL |
| SEQ ID NO: 31 | MGVLKFKHIFFRSFVKSSGVSQIVFTFLLIPCCLTLNFRAPPV |
| | IPNAPFLWAWNAPSEFCLGKFDEPLDMSLESFIGSPRINATGQ |
| | GVTIFYVDRLGYYPYIDSITGVTVNGGIPQKISLQDHLDKAKK |
| | DITFYMPVDNLGMAVIDWEEWRPTWARNWKPKDVYKNRSIELV |
| | QQQNVQLSLTEATEKAKQEFEKAGKDFLVETIKLGKLLRPNHL |
| | WGYYLFPDCYNHHYKKPGYNGSCFNVEIKRNDDLSWLWNESTA |
| | LYPSIYLNTQQSPVAATLYVRNRVREAIRVSKIPDAKSPLPVF |
| | AYTRIVFTDQVLKFLSQDELVYTFGETVALGASGIVIWGTLSI |
| | MRSMKSCLLLDNYMETILNPYIINVTLAAKMCSQVLCQEQGVC |
| | IRKNWNSSDYLHLNPDNFAIQLEKGGKFTVRGKPTLEDLEQFS |
| | EKFYCSCYSTLSCKEKADVKDTDAVDVCIADGVCIDAFLKPPM |
| | ETEEPQIFYNASPSTLSATMFIVSILFLIISSVASL |
| SEQ ID NO: 32 | LNFRAPPVIPNAPELWAWNAPSEFCLGKFDEPLDMSLESFIGS |
| | PRINATGQGVTIFYVDRLGYYPYIDSITGVTVNGGIPQKISLQ |
| | DHLDKAKKDITFYMPVDNLGMAVIDWEEWRPTWARNWKPKDVY |
| | KNRSIELVQQQNVQLSLTEATEKAKQEFEKAGKDFLVETIKLG |
| | KLLRPNHLWGYYLFPDCYNHHYKKPGYNGSCFNVEIKRNDDLS |
| | WLWNESTALYPSIYLNTQQSPVAATLYVRNRVREAIRVSKIPD |
| | AKSPLPVFAYTRIVFTDQVLKELSQDELVYTFGETVALGASGI |
| | VIWGTLSIMRSMKSCLLLDNYMETILNPYIINVTLAAKMCSQV |
| | LCQEQGVCIRKNWNSSDYLHLNPDNFAIQLEKGGKFTVRGKPT |
| | LEDLEQFSEKFYCSCYSTLSCKEKADVKDTDAVDVCIADGVCI |
| | DAFLKPPMETEEPQIFYNASPSTLSATMFIVSILFLIISSVAS |
| | L |

In some embodiments, the subcutaneous composition can comprise:
about 144 mg/mL to about 176 mg/mL of the bispecific EGFR/c-Met antibody;
about 10 mM to about 50 mM of acetate and/or a pharmaceutically acceptable acetate salt;
about 6.8% (w/v) to about 10.2% (w/v) of sucrose;
about 0.036% (w/v) to about 0.084% (w/v) of polysorbate 80 (PS80);
about to 0.8 mg/mL to about 1.2 mg/mL of methionine;
about 16 μg/mL to about 24 μg/mL of ethylenediaminetetraacetic acid (EDTA); and
about 2,000 U/mL of the hyaluronidase, wherein the composition has a pH from about 5.2 to about 6.2.

In some embodiments, the subcutaneous composition comprises:
about 160 mg/mL of the bispecific antibody, wherein the HC1 of the bispecific antibody comprises the amino acid sequence of SEQ ID NO: 17, the LC1 of the bispecific antibody comprises the amino acid sequence of SEQ ID NO: 18, the HC2 of the bispecific antibody comprises the amino acid sequence of SEQ ID NO: 19, and the LC2 of the bispecific antibody comprises the amino acid sequence of SEQ ID NO: 20,
about 30 mM acetate and/or pharmaceutically acceptable acetate salt,
about 8.5% sucrose,
about 1 mg/mL L-methionine
polysorbate 80 to a final concentration of about 0.06% (w/v)
EDTA to a final concentration of about 20 μg/mL,
rHuPH20 to a final concentration of about 2,000 U/mL, and
a pH of about 5.7.

In some embodiments, the subcutaneous composition comprises:
about 160 mg/mL of the bispecific antibody, wherein the HC1 of the bispecific antibody comprises the amino acid sequence of SEQ ID NO: 17, the LC1 of the bispecific antibody comprises the amino acid sequence of SEQ ID NO: 18, the HC2 of the bispecific antibody comprises the amino acid sequence of SEQ ID NO: 19, and the LC2 of the bispecific antibody comprises the amino acid sequence of SEQ ID NO: 20, wherein Asp99 of SEQ ID NO: 17 is isomerized, wherein the residue numbering starts from N-terminus of SEQ ID NO: 17,
about 30 mM acetate and/or pharmaceutically acceptable acetate salt,
about 8.5% sucrose,
about 1 mg/mL L-methionine
polysorbate 80 to a final concentration of about 0.06% (w/v)
EDTA to a final concentration of about 20 μg/mL,
rHuPH20 to a final concentration of about 2,000 U/mL, and
a pH of about 5.7.

In some embodiments, the subcutaneous composition comprises:
about 160 mg/mL of the bispecific antibody, wherein the HC1 of the bispecific antibody comprises the amino acid sequence of SEQ ID NO: 17, the LC1 of the bispecific antibody comprises the amino acid sequence of SEQ ID NO: 18, the HC2 of the bispecific antibody comprises the amino acid sequence of SEQ ID NO: 19, and the LC2 of the bispecific antibody comprises the amino acid sequence of SEQ ID NO: 20, wherein Asp99 of SEQ ID NO: 17 is isomerized, wherein the residue numbering starts from N-terminus of SEQ ID NO: 17,
and wherein the composition further comprises a non-modified EGFR/c-Met binding agent comprising first polypeptide comprising SEQ ID NO: 17, a second polypeptide comprising SEQ ID NO: 18, a third polypeptide comprising SEQ ID NO: 19, and a fourth polypeptide comprising SEQ ID NO: 20;
about 30 mM acetate and/or pharmaceutically acceptable acetate salt,
about 8.5% sucrose,
about 1 mg/mL L-methionine
polysorbate 80 to a final concentration of about 0.06% (w/v)
EDTA to a final concentration of about 20 μg/mL,
rHuPH20 to a final concentration of about 2,000 U/mL, and
a pH of about 5.7.

### 6.6 Pharmaceutical Compositions

In another general aspect, provided is a pharmaceutical composition comprising the EGFR/c-Met binding agent provided herein and a pharmaceutically acceptable excipient.

In another general aspect, provided herein is a method of producing a pharmaceutical composition comprising a binding agent or an antigen binding region thereof provided herein, comprising combining a binding agent or an antigen binding region thereof with a pharmaceutically acceptable carrier to obtain the pharmaceutical composition.

According to particular embodiments, the pharmaceutical compositions described herein are formulated to be suitable for the intended route of administration to a subject. For example, the pharmaceutical compositions described herein can be formulated to be suitable for intravenous, subcutaneous, or intramuscular administration.

### 6.7 Kits

In another general aspect, the disclosure relates to kits comprising an isolated binding agent or antigen binding region thereof provided herein and instructions for use.

In one embodiment, provided is a kit comprising the EGFR/c-Met binding agent provided herein. The described kits can be used to carry out the methods of using the EGFR/c-Met binding fragments provided herein, or other methods known to those skilled in the art. In some embodiments, the described kits can include the binding proteins described herein and reagents for use in detecting the presence of a target antigen (e.g., EGFR and c-Met) in a biological sample. Accordingly, the described kits can include one or more of the binding agents (e.g., antibodies or proteins), or an antigen binding region(s) thereof, described herein and a vessel for containing the binding agent or antigen binding region when not in use, instructions for use of the antibody or fragment, the binding agent or antigen binding region affixed to a solid support, and/or detectably labeled forms of the binding agent or antigen binding region, as described herein.

In another embodiment, provided is a kit comprising the EGFR/c-Met binding agent comprising a first binding region specifically binding EGFR and a second binding region specifically binding c-Met provided herein.

In some embodiments, the kit comprises an antibody described herein and reagents for detecting the binding agent. The kit can further include one or more other elements including: instructions for use; other reagents, e.g., a label, a therapeutic agent, or an agent useful for chelating, or otherwise coupling, an antibody to a label or therapeutic agent, or a radioprotective composition; devices or other materials for preparing the antibody for administration; pharmaceutically acceptable carriers; and devices or other materials for administration to a subject.

In some embodiments, the kit comprises the EGFR/c-Met binding agent provided herein in a container and instructions for use of the kit.

In some embodiments, the EGFR/c-Met binding agent in the kit is labeled.

In case of conflict, the specification, including definitions, will control. As used herein and in the appended claims, the singular forms “a,” “an,” and “the” include plural referents unless the context clearly dictates otherwise. Thus, for example, reference to “a peptide sequence” or “a treatment,” includes a plurality of such sequences, treatments, and so forth. It is further noted that the claims can be drafted to exclude any optional element. As such, this statement is intended to serve as antecedent basis for use of such exclusive terminology such as “solely,” “only” and the like in connection with the recitation of claim elements, or use of a “negative” limitation.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges can independently be included in the smaller ranges, and are also encompassed within the invention, subject to any specifically excluded limit in the stated range. Where the stated range includes one or both of the limits, ranges excluding either or both of those included limits are also included in the invention.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges, unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 96%, 97%, etc., as well as 91.1%, 91.2%, 91.3%, 91.4%, 91.5%, etc., 92.1%, 92.2%, 92.3%, 92.4%, 92.5%, etc., and so forth. In addition, reference to a range of 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250 includes 1, 2, 3, 4, 5, 6, 7, 8, 9, 10, 11, 12, 13, 14, 15, 16, 17, 18, 19, 20, etc. In a further example, reference to a range of 25-250, 250-500, 500-1000, 1000-2500, 2500-5000, 5000-25,000, or 5000-50,000 includes any numerical value or range within or encompassing such values, e.g., 25, 26, 27, 28, 29 . . . 250, 251, 252, 253, 254 . . . 500, 501, 502, 503, 504 . . . , etc. The use of a series of ranges includes combinations of the upper and lower ranges to provide another range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, includes ranges such as 5-20, 5-30, 5-40, 5-50, 5-75, 5-100, 5-150, and 10-30, 10-40, 10-50, 10-75, 10-100, 10-150, and 20-40, 20-50, 20-75, 20-100, 20-150, and so forth.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

Particular embodiments of this invention are described herein, including the best mode known to the inventors for carrying out the invention. Upon reading the foregoing description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All publications, patent applications, accession numbers, and other references cited in this specification are herein incorporated by reference in its entirety as if each individual publication or patent application were specifically and individually indicated to be incorporated by reference. The publications discussed herein are provided solely for their disclosure prior to the filing date of the present application. Nothing herein is to be construed as an admission that the present invention is not entitled to antedate such publication by virtue of prior invention. Further, the dates of publication provided can be different from the actual publication dates which can need to be independently confirmed.

A number of embodiments of the invention have been described. Nevertheless, it will be understood that various modifications may be made without departing from the spirit and scope of the invention. Accordingly, the descriptions in the Experimental section are intended to illustrate but not limit the scope of invention described in the claims.

### EMBODIMENTS

1. A composition comprising:
   a variant EGFR/c-Met binding agent comprising a first polypeptide comprising SEQ ID NO: 17, a second polypeptide comprising SEQ ID NO: 18, a third polypeptide comprising SEQ ID NO: 19, and a fourth polypeptide comprising SEQ ID NO: 20,
   wherein Asp99 of SEQ ID NO: 17 is isomerized,
   wherein the residue numbering starts from N-terminus of SEQ ID NO: 17,
   and a hyaluronidase.
2. The composition of embodiment 1, wherein the composition further comprises a non-modified EGFR/c-Met binding agent comprising first polypeptide comprising SEQ ID NO: 17, a second polypeptide comprising SEQ ID NO: 18, a third polypeptide comprising SEQ ID NO: 19, and a fourth polypeptide comprising SEQ ID NO: 20.
3. The composition of embodiment 2, wherein the composition facilitates binding to any one or more of EGFR and c-Met.
4. The composition of any one of embodiments 1-3, wherein the Asp99 isomerized variant of the EGFR/c-Met binding agent comprises from more than about 0% to about 10% of the EGFR/c-Met binding agents in the composition.
5. The composition of any one of embodiments 1-4, wherein the Asp99 isomerized variant EGFR/c-Met binding agent and the non-modified EGFR/c-Met binding agent have the fucose content between about 0% and about 20%.
6 The composition of any one of embodiments 1-5, wherein the hyaluronidase is a PH20.
7. The composition of embodiment 6, wherein the PH20 comprises an amino acid sequence selected from the group consisting of SEQ ID NO: 21-32.
8. The composition of embodiment 7, wherein the PH20 comprises the amino acid sequence of SEQ ID NO: 21.
9. The composition of embodiment 7, wherein the PH20 comprises the amino acid sequence of SEQ ID NO: 22.
10. The composition of embodiment 7, wherein the PH20 comprises the amino acid sequence of SEQ ID NO: 23.
11. The composition of embodiment 7, wherein the PH20 comprises the amino acid sequence of SEQ ID NO: 24.
12. The composition of embodiment 7, wherein the PH20 comprises the amino acid sequence of SEQ ID NO: 25.
13. The composition of embodiment 7, wherein the PH20 comprises the amino acid sequence of SEQ ID NO: 26.
14. The composition of embodiment 7, wherein the PH20 comprises the amino acid sequence of SEQ ID NO: 27.
15. The composition of embodiment 7, wherein the PH20 comprises the amino acid sequence of SEQ ID NO: 28.
16. The composition of embodiment 7, wherein the PH20 comprises the amino acid sequence of SEQ ID NO: 29.
17. The composition of embodiment 7, wherein the PH20 comprises the amino acid sequence of SEQ ID NO: 30.
18. The composition of embodiment 7, wherein the PH20 comprises the amino acid sequence of SEQ ID NO: 31.
19. The composition of embodiment 7, wherein the PH20 comprises the amino acid sequence of SEQ ID NO: 32.
20. A pharmaceutical composition comprising a composition of any one of embodiments 1-19 and a pharmaceutically acceptable carrier.
21. A method of treating a cancer or tumor in a subject in need thereof, comprising administering an effective amount of the composition of embodiment 20.
22. A method for treating a subject having a EGFR or c-Met-expressing cancer or tumor, comprising administering or providing for administration of an effective amount of the composition of embodiment 20 to the subject.
23. The method of embodiment 21, wherein the cancer or tumor is selected from lung cancer, colorectal cancer, and head and neck cancer.

### 7. EXAMPLES

The following is a description of various methods and materials used in the studies, and are put forth so as to provide those of ordinary skill in the art with a complete disclosure and description of how to make and use the present invention, and are not intended to limit the scope of what the inventors regard as their invention nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present invention. Efforts have been made to ensure accuracy with respect to numbers used (e.g., amounts, temperature, etc.), but some experimental errors and deviations should be accounted for.

### 7.1 Example 1: Preparation and Characterization of a Bispecific Antibody Composition

The anti-EGFR monospecific antibodies and anti-c-Met monospecific antibodies were expressed in two CHO cell lines, each cell line having reduced fucosylation ability resulting in bispecific antibodies with reduced fucose content in the antibody polysaccharide chain, and hence have a fucosyl content between about 0% and about 20%.

An inoculum of Chinese Hamster Ovary cells stably transfected with expression vector constructs comprising the nucleic acid sequences encoding the anti-EGFR arm heavy chain 1 of SEQ ID NO: 17, and the anti-EGFR arm light chain 1 of SEQ ID NO: 18, was cultured in either 2000 L or 15000 L bioreactors containing a liquid cell culture medium.

An inoculum of Chinese Hamster Ovary cells stably transfected with expression vector constructs comprising the nucleic acid sequences encoding the anti-c-Met arm heavy chain 2 of SEQ ID NO: 19, and the anti-c-Met arm light chain 2 of SEQ ID NO: 20, was cultured in either 2000 L or 15000 L bioreactors containing a liquid cell culture medium.

Bioreactors were operated at a temperature of about 34.5° C. to about 37.5° C. Air and oxygen were sparged into the culture medium and a pH of about 6.75 to 7.25 was maintained. Clarified cell culture supernatant was harvested by centrifugation and filtration of the cell culture medium. This clarified supernatant was then subjected to protein A chromatography and impurities were allowed to flow off this chromatography column. Bound protein including monospecific antibody molecules was then eluted from the protein A column and followed by filtration.

Bispecific EGFR/c-Met antibodies were generated by combining a monospecific EGFR mAb and a monospecific c-Met mAb in in vitro Fab arm exchange (as described in WO2011/131746, incorporated by reference herein in its entirety). Briefly, at about 1-20 mg/ml at a molar ratio of 1:1 of each antibody in PBS, pH 7-7.4 and 75 mM 2-mercaptoethanolamine (2-MEA) was mixed together and incubated at 25-37° C. for 2-6 h, followed by removal of the 2-MEA via dialysis, diafiltration, tangential flow filtration and/or spinned cell filtration using standard methods.

The bispecific EGFR/c-Met antibodies were further purified after the in vitro Fab-arm exchange using hydrophobic interaction chromatography to minimize residual parental c-Met and EGFR antibodies using standard methods.

The EGFR/c-Met bispecific antibodies were reconstituted to the final concentration of 160 mg/mL in 30 mM acetate, 8.5% [w/v] sucrose, 0.06% [w/v], polysorbate 80, 1 mg/mL methionine, and 20 μg/mL EDTA, pH 5.7, designated as drug substance (DS).

The drug product (DP) composition included EGFR/c-Met bispecific antibodies at the concentration of 160 mg/mL in 30 mM acetate, 8.5% [w/v] sucrose, 0.06% [w/v], polysorbate 80, 1 mg/mL methionine, 20 μg/mL EDTA, and 2000 IU rHuPH20, pH 5.7, designated as drug product (DP).

Reference Material (RM) was produced by diluting drug substance (DS) to 50 mg/mL with formulation buffer, was aliquoted in 0.5 mL polypropylene cryovials at a fill volume of approximately 0.25 mL and stored at ≤−60° C. Thawed RM was further characterized.

EGFR HC1 Asp99 isomerization was detected in the CDR3 of the anti-EGFR arm. EGFR HC1 Asp99 isomerization was measured by a multi-attribute monitoring (MAM) peptide mapping method using reversed phase ultra high-performance liquid chromatography (UHPLC) and on-line mass spectrometry. This post-translational modification (PTM) was quantitated by peak area integration of the Selected Ion Recording (SIR), and the results were reported as percentages of total peak area of the modified and unmodified peptides.

EGFR HC1 Asp99 isomerization was determined to impact EGFR binding function of the EGFR/c-Met bispecific antibodies, as demonstrated using a homogeneous competitive time resolved fluorescence resonance energy transfer (TR-FRET) assay format. In this procedure, varying concentrations of unlabeled bispecific EGFR-c-Met antibody sample compete with donor fluorophore (Europium (Eu) chelate) labeled bispecific EGFR-c-Met antibody for binding to an acceptor fluorophore (Cy5)-labeled EGFR antigen. Excitation of the donor fluorophore results in a transfer of energy to the bound acceptor fluorophore (FRET process). The resultant FRET is detected by emission of light at 665 nm using a microplate reader capable of measuring time-resolved fluorescence. Sample dose response curves were compared to the RM.

EGFR HC1 Asp99 isomerization was determined to impact EGFR bioactivity, for example ADCC of the EGFR/c-Met bispecific antibodies, as demonstrated using a gene reporter kit, which used engineered Jurkat cells that expressed the FcγRIIIa receptor. The potency was calculated relative to the EGFR/c-Met bispecific antibodies reference material (RM).

1. A composition comprising:
a variant EGFR/c-Met binding agent comprising a first polypeptide comprising SEQ ID NO: 17, a second polypeptide comprising SEQ ID NO: 18, a third polypeptide comprising SEQ ID NO: 19, and a fourth polypeptide comprising SEQ ID NO: 20, wherein Asp99 of SEQ ID NO: 17 is isomerized,
wherein the residue numbering starts from N-terminus of SEQ ID NO: 17, and a hyaluronidase.

2. The composition of claim 1, wherein the composition further comprises a non-modified EGFR/c-Met binding agent comprising first polypeptide comprising SEQ ID NO: 17, a second polypeptide comprising SEQ ID NO: 18, a third polypeptide comprising SEQ ID NO: 19, and a fourth polypeptide comprising SEQ ID NO: 20.

3. The composition of claim 2, wherein the composition facilitates binding to any one or more of EGFR and c-Met.

4. The composition of claim 1, wherein the Asp99 isomerized variant of the EGFR/c-Met binding agent comprises from more than about 0% to about 10% of the EGFR/c-Met binding agents in the composition.

5. The composition of claim 1, wherein the Asp99 isomerized variant EGFR/c-Met binding agent and the non-modified EGFR/c-Met binding agent have the fucose content between about 0% and about 20%.

6. The composition of claim 1, wherein the hyaluronidase is a PH20.

7. A pharmaceutical composition comprising the composition of claim 1 and a pharmaceutically acceptable carrier.

8. A method of treating a cancer or tumor in a subject in need thereof, comprising administering an effective amount of the composition of claim 7.

9. A method for treating a subject having a EGFR or c-Met-expressing cancer or tumor, comprising administering or providing for administration of an effective amount of the composition of claim 7 to the subject.

10. The method of claim 9, wherein the cancer or tumor is selected from lung cancer, colorectal cancer, and head and neck cancer.


SEQUENCE LISTING (32 sequences)

SEQ ID NO: 1 - protein, 5 aa, Homo sapiens
TYGMH

SEQ ID NO: 2 - protein, 17 aa, Homo sapiens
VIWDDGSYKYYGDSVKG

SEQ ID NO: 3 - protein, 16 aa, Homo sapiens
DGITMVRGVMKDYFDY

SEQ ID NO: 4 - protein, 11 aa, Homo sapiens
RASQDISSALV

SEQ ID NO: 5 - protein, 7 aa, Homo sapiens
DASSLES

SEQ ID NO: 6 - protein, 9 aa, Homo sapiens
QQFNSYPLT

SEQ ID NO: 7 - protein, 5 aa, Homo sapiens
SYGIS

SEQ ID NO: 8 - protein, 17 aa, Homo sapiens
WISAYNGYTNYAQKLQG

SEQ ID NO: 9 - protein, 10 aa, Homo sapiens
DLRGTNYFDY

SEQ ID NO: 10 - protein, 11 aa, Homo sapiens
RASQGISNWLA

SEQ ID NO: 11 - protein, 7 aa, Homo sapiens
AASSLLS

SEQ ID NO: 12 - protein, 9 aa, Homo sapiens
QQANSFPIT

SEQ ID NO: 13 - protein, 125 aa, Homo sapiens [sequence omitted]

SEQ ID NO: 14 - protein, 107 aa, Homo sapiens [sequence omitted]

SEQ ID NO: 15 - protein, 119 aa, Homo sapiens
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSS

SEQ ID NO: 16 - protein, 107 aa, Homo sapiens
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIK

SEQ ID NO: 17 - protein, 455 aa, Homo sapiens
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 18 - protein, 214 aa, Homo sapiens
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 449 aa, Homo sapiens
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 20 - protein, 214 aa, Homo sapiens
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 443 aa, Homo sapiens
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEP

SEQ ID NO: 22 - protein, 447 aa, Homo sapiens [sequence omitted]

SEQ ID NO: 23 - protein, 446 aa, Homo sapiens
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIF

SEQ ID NO: 24 - protein, 445 aa, Homo sapiens
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQI

SEQ ID NO: 25 - protein, 444 aa, Homo sapiens
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQ

SEQ ID NO: 26 - protein, 509 aa, Homo sapiens
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 27 - protein, 474 aa, Homo sapiens
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 28 - protein, 509 aa, Homo sapiens [sequence omitted]

SEQ ID NO: 29 - protein, 474 aa, Homo sapiens
LNFRAPPVIPNVAFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSIWFLIISSVASL

SEQ ID NO: 30 - protein, 509 aa, Homo sapiens
MGVLQFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 31 - protein, 509 aa, Homo sapiens
MGVLKFKHIFFRSFVKSSGVSQIVFTFLLIPCCLTLNFRAPPVIPNAPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 32 - protein, 474 aa, Homo sapiens
LNFRAPPVIPNAPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL